(19) United States
(12) Patent Application Publication
(10) Pub. No.: US 2005/0064583 A1
(43) Pub. Date: Mar. 24, 2005

(54) Temperature controlled illuminator for treating biological samples

(76) Inventors: Frank Caruso, Golden, CO (US); Carl Schmidt, Lakewood, CO (US); Kevin Klimek, Denver, CO (US)

Correspondence Address: Greenlee, Winner and Sullivan, Suite 201, 5370 Manhattan Circle, Boulder, CO, 80303, US

(21) Appl. No.: 10/947,855

(22) Filed: Sep. 22, 2004

Related U.S. Application Data
(60) Provisional application No. 60/506,020, filed on Sep. 24, 2003.

(51) Int. Cl. C12Q 1/68; C12M 1/34; G02B 6/06

(52) U.S. Cl. 435/287.2; 362/572; 435/6

(57) ABSTRACT

Methods, devices and device components are presented for reducing pathogenic biological contaminants in biological samples, in particular, methods, devices and device components for treating biological samples with electromagnetic radiation. In one aspect, the invention provides illuminators having differentially cooled light sources which exhibit improved light source longevity over conventional high intensity illuminators. In another aspect, the invention provides illuminators having closed loop feedback temperature control which produce radiant intensities and powers that are substantially constant as a function of time.

### CROSS-REFERENCE TO RELATED APPLICATIONS

This application claims priority under 35 U.S.C. 119(e) to provisional patent application 60/506,020, filed Sep. 24, 2003, which is hereby incorporated by reference in its entirety to the extent not inconsistent with the disclosure herein.

### BACKGROUND OF INVENTION

Collection, processing and purification of biological samples are important processes in a range of medical therapies and procedures. Important biological samples used as therapeutic agents include whole blood and its various purified blood components, such as red blood cells, white blood cells and plasma. In the field of transfusion medicine, one or more whole blood components are directly introduced into a patient's blood stream to replace a depleted or deficient component. Infusion of plasma-derived materials, such as blood proteins, also plays a critical role in a number of therapeutic applications. For example, plasma-derived immunoglobulin is commonly provided to supplement a patient's compromised immune system. Due to increases in the demand for purified biological samples for transfusion, infusion and transplantation therapies, substantial research efforts have been directed at improving the availability, safety and purity of biological samples used as therapeutic agents.

The safety of transfusion, infusion and transplantation therapies is dependent on identifying the presence of and reducing pathogenic biological contaminants, such as viruses, bacteria, fungi, bacteriphages and protozoa, present in donated biological samples. The presence of pathogens in samples used as therapeutic agents is dangerous as these contaminants can infect patients undergoing treatment and deleteriously affect recovery time, quality of life and future health. Further, the presence of pathogenic contaminants in biological samples is of serious consequence not only to patients undergoing therapeutic transfusion, infusion and transplantation procedures, but also to doctors and other hospital personnel who handle, process and administer these materials.

While biological samples used as therapeutic agents are currently safer than ever, the risk of exposure to pathogens in human blood reservoirs remains significant. A large number of deleterious contaminants have been identified in intracellular and extracellular fractions of human blood. For example, it is estimated that approximately 1 in 34,000 donated blood component samples are contaminated with human immunodeficiency virus type I/II (HIV), hepatitis B and C (HVB and HVC) or human T-lymphotropic virus type I/II (HTLV I/II). In addition, it has also been demonstrated that human blood reservoirs are routinely contaminated with other pathogens which are not assayed in typical blood screening protocols, such as transfusion-transmitted virus, hepatitis G virus, human herpes virus 8, HTLV-2, west Nile virus, hepatitis A, TT virus, SEN-V malaria, babesia, trypanosome, and parvo B19 virus. As a result of the risks associated with these contaminants, blood components are currently underutilized as effective therapeutic agents.

Over the last decade, a number of methods have been developed for reducing the risks associated with pathogenic biological contaminants in biological samples, especially donated blood component samples. Screening of donors and acquired blood samples has been demonstrated to provide an effective method for identifying and avoiding pathogen-contaminated biological samples. Effective screening methods combine rigorous donor interviews and pathogen specific assay techniques. Despite the reduction in the transmission of pathogens achieved by screening, these methods remain susceptible to problems associated with the presence of pathogenic contaminants. First, a measurable incidence of pathogen transmission is associated with screened blood samples due to the difficulty of detecting pathogens which are capable of causing infection present at very low levels. Second, blood sample screening results in the disposal of large quantities of donated blood which are deemed unusable. As the supply of donated blood is finite, disposal of contaminated blood significantly reduces the availability of blood components needed for important therapeutic procedures. Third, current screening methodologies are limited to approximately nine pathogen-specific assays. Accordingly, a number of pathogens known to be present in blood samples are not currently assayed, not to mention those blood pathogens present in human blood which have yet to be identified. Finally, screening methods are costly and labor intensive, requiring the expenditure great deal of resources to be performed effectively.

A different approach to reducing the risks associated with pathogenic contamination of biological samples involves decreasing the biological activity of pathogens present in biological samples by directly killing the pathogens or rendering them incapable of replication. Over the last decade, a variety of methods for reducing pathogens in blood samples have emerged including direct photoreduction, the use of detergents for inactivating viruses having lipid membranes, chemical treatment methods and photoinduced chemical reduction techniques. Due to its compatibility with high-volume pathogen inactivation and demonstrated efficacy, photoinduced chemical reduction and direct photoreduction have emerged as especially promising techniques for treating biological samples. U.S. Pat. Nos. 6,277,337, 5,607,924, 5,545,516, 4,915,683, 5,516,629, and 5,587,490 describe exemplary applications of photoinduced chemical reduction and direct photoreduction methods for inactivating of pathogens in blood.

In photoinduced chemical reduction methods, effective amounts of one or more nontoxic, photosensitive compounds are added to a biological sample, which is subsequently illuminated with electromagnetic radiation. Illumination activates the photosensitive compounds, thereby initiating one or more chemical reactions which kill the pathogens present in the sample or substantially prevents the pathogens from replicating. In contrast, illumination alone results in destruction or inactivation of the pathogens in direct photoreduction methods. Photoinduced chemical reduction methods are useful in some applications to direct photoreduction because these techniques are often compatible with radiant wavelengths and intensities which do not significantly damage and/or affect the biological activity of therapeutic cellular and noncellular components of a sample undergoing treatment. Particularly, use of a photosensitive compound can avoid exposure of a biological sample to high intensities of ultraviolet light, which can jeopardize the viability of healthy cells and tissue.

Effective photoinduced chemical reduction requires achieving and maintaining optimal illumination and sample conditions. First, the wavelength of the activating light must be within the absorption range of the photosensitive compound(s), preferably for some applications close to the absorbance maximum, to provide efficient photoactivation. Second, the photosensitive compounds must be well mixed throughout the sample. Third, the radiant intensities provided to a sample undergoing pathogen reduction must be large enough to excite a significant population of photosensitive reagents in the sample. Fourth, illumination conditions must be substantially constant as a function of time to ensure sufficient exposure to electromagnetic radiation and to determine the sample irradiation time required to achieve a desired extent of pathogen reduction.

Despite the demonstrated efficacy of photoinduced chemical reduction and direct photo reduction, realization of the full benefits of these techniques to blood processing remains unfulfilled due to problems associated with achieving reproducible, uniform and constant sample irradiation conditions. These problems are especially pronounced under the lamp conditions necessary to generate the high intensity irradiation conditions required for effective pathogen inactivation. For example, light sources employed for sample irradiation typically exhibit substantial variations in radiant intensities and wavelength distributions during irradiation, which prevent calculation or measurement of pathogen reduction rates achieved during treatment. Such variations impede accurate determination of exposure times required to achieve a desired extent of pathogen inactivation. The ability to accurately predict and/or monitor the extent of inactivation achieved during photochemical processing is essential to ensure that the treated sample is substantially free from biologically active pathogens and safe for use as a therapeutic agent.

The nature and design of light sources chosen for illumination greatly affect the effectiveness of pathogen reduction achieved. Tubular fluorescent lamps are commonly used for sample illumination because they provide an elongated light-emitting surface well-suited for illumination of biological samples, such as static and flowing blood and/or blood components. These lamps operate by passing an electric current through a tube having an internal phosphor coating which is filled with mercury vapor. The current generates a stream of electrons which excite electronic transitions in the mercury atoms via collisions. Excited mercury atoms release this energy in the form of ultraviolet light, which subsequently excites the internal phosphor coating causing it to emit visible light. The design of most conventional fluorescent lamps includes first and second cathodes positioned at the ends of the fluorescent bulbs, which generate the electric discharge through the tube. The presence of first and second cathodes, however, also results in a significant temperature gradient across the length of the fluorescent lamp. For example, a typical tubular fluorescent bulb operating in open air has a lamp surface temperature gradient of up to approximately 30° C. along its longitudinal axis, wherein the regions of the lamp proximate to the cathodes are hotter than the lamp center.

Although tubular fluorescent lamps are capable of providing the radiant intensities and wavelengths required for direct photoreduction and photoinduced chemical reduction methods, these light sources are susceptible to problems which undermine their use in the treatment of biological samples. First, these lamps exhibit substantial intensity fluctuations during illumination which can result in underexposure of a biological sample to electromagnetic radiation. For example, changes in ambient temperature often directly affect lamp temperature, which in turn influences the radiant intensities and the distribution of radiant wavelengths delivered to a sample. In addition, variation of radiant intensity and wavelength distribution degrades the reproducibility of sample irradiation conditions and impedes calculation of the illumination time required to achieve a desired level of pathogen reduction. Second, temperature gradients generated by these lamps substantially reduce their effective lifetime. The existence of higher temperature regions proximate to lamp cathodes increases the rates of electrode degradation and phosphor degradation in the lamp, thereby decreasing lamp longevity and increasing the overall cost of pathogen reduction procedures. Moreover, temperature gradients in these lamps contribute to observed variations in radiant intensity and wavelength distribution during sample illumination. Specifically, temperature gradients increase the rate at which the output of a fluorescent lamp decreases as a function of time. This decrease in lamp output can result in sample underexposure and impedes accurate characterization of the radiant energies delivered to a sample during illumination.

To address the problems associated with the use of tubular fluorescent lamps for pathogen reduction, differential cooling methods have emerged in recent years which are reported to reduce temperature gradients observed in these lamps. Several of these methods utilize heat sink devices which conduct heat away from specific, high energy-producing regions of a lamp, such as the cathode regions. For example, U.S. Pat. No. 6,417,832 describes a back light assembly having differential cooling for a flat screened LCD display panel wherein heat sinks are attached to the cathode regions of the fluorescent lamps to reduce the temperature gradient observed along the longitudinal axis of each fluorescent lamp. Although the reference reports a reduction in the temperature gradient observed between cathode and center regions, the disclosed method requires establishing reliable thermal contact between the heat sink and the glass exterior surface of the lamp. Such thermal contact is difficult to achieve reproducibly and typically requires the use of thermal tape or thermally conductive grease. Moreover, the disclosed differential cooling system is inconvenient because thermal contact between the heat sink and lamp bulb must be reproducibly reestablished each time the fluorescent lamp bulbs are replaced or serviced.

Alternatively, methods utilizing forced convection to preferentially cool high energy-producing regions of a fluorescent lamp have been reported to reduce observed temperature gradients. For example, U.S. Pat. No. 6,223,071 describes an illuminator used for photodynamic therapy and diagnosis reportedly providing differential cooling using a plenum enclosing a U-shaped fluorescent bulb. The plenum is equipped with a plurality of intake vents proximate to free ends of the straight segments of the U-shaped lamp and a plurality of exhaust vents proximate to the arcuate regions of the lamp. The exhaust vents are equipped with fans which draw a flow of ambient air through the lamp chamber. This configuration is reported to achieve greater cooling at the ends of the lamp than the center arcuate lamp region. U.S. Pat. No. 6,223,071 is limited, however, to methods or devices for cooling U-shaped fluorescent bulbs and it is unclear if the differential cooling method disclosed would be compatible with fluorescent lamps having different shapes, such as linear tubular lamps. In addition, the forced convection cooling system disclosed is incapable of providing discrete control of cooling flow rates provided to the fluorescent lamp surface as a function of distance along the length of the lamp. Rather, the differential cooling system described is only capable of generating a single differential profile of cooling flows to the lamp surface.

It will be appreciated from the foregoing that a need exists for methods and devices for providing electromagnetic radiation to biological samples. Particularly, illuminators capable of providing substantially constant radiant intensities and powers throughout a selected sample treatment time are needed.

### SUMMARY OF THE INVENTION

The present invention provides illuminators for direct photoreduction or photoinduced chemical reduction of pathogens in biological samples, such as blood and blood component samples, which ensure that a biological sample undergoing treatment is sufficiently and uniformly exposed to electromagnetic radiation. In addition, the present invention provides illuminators having reduced temperature gradients, which provide extended useful lamp lifetimes.

This invention provides methods, devices and device components for treating biological samples with electromagnetic radiation, such as whole blood, blood components, blood products and tissue samples. It is an object of the present invention to provide methods and devices for treating a biological sample so that it is safe for use as a therapeutic agent. It is further an object of the present invention to provide methods and devices for treating a biological sample so that the sample is safe for handling, processing and/or administering to patients. It is yet another object of the present invention to provide methods and devices for treating a biological sample so that it is safe for transfusion or transplantation into a patient.

In one aspect, the present invention comprises methods, devices and device components for reducing pathogenic contaminants, such as viruses, bacteria, fungi, bacteriophages and protozoa, present in a biological sample. Illuminators are presented which provide electromagnetic radiation for directly reducing pathogenic contaminants in biological samples by photoreduction. Alternatively, illuminators are presented which provide electromagnetic radiation for indirectly reducing pathogenic contaminants in biological samples by inducing photochemical reactions between at least one photosensitive compound present in the sample and the pathogens. Further, the present invention provides illuminators for reducing pathogenic contaminants via a combination of direct photoreduction and inducing photochemical reactions involving one or more photosensitive compounds. Exemplary methods and devices of the present invention provide electromagnetic radiation for reducing pathogenic contaminants which does not substantially affect the biological activity or viability of one or more therapeutic components of a biological sample.

In another aspect, the present invention comprises methods, devices and device components for reducing the activity of leukocytes present in a biological sample, such as a whole blood sample or blood component sample. Reducing the activity of leukocytes is desirable when suppression of immune response or autoimmune response is required. For example, reduction of leukocyte activity is beneficial in processes involving transfusion of red blood cells, platelets and/or plasma when patient or donor leukocytes are present. Illuminators are presented which provide electromagnetic radiation for reducing leukocyte activity in biological samples by inducing photochemical reactions between at least one photosensitive compound and at least a portion of the leucocytes present. Exemplary methods and devices of the present invention provide electromagnetic radiation for reducing the biological activity of leukocytes which does not substantially affect the biological activity or viability of therapeutic components of a biological sample.

In yet another aspect, the present invention provides illuminators having fluorescent lamps or other light sources which exhibit temperature gradients less than those observed in conventional high intensity illuminators, such as illuminators used for UV curing of polymers or water purification. Illuminators of this aspect of the present invention are beneficial because they maximize the useful lifetimes of fluorescent lamps or other light sources and, thereby, reduce the overall cost of direct photoreduction methods or photoinitiated chemical reduction methods. Illuminators of the present invention having reduced lamp temperature gradients require less maintenance than conventional high intensity illuminators. In addition, illuminators of this aspect of the invention also provide more uniform radiant intensities and radiant powers as a function of time by minimizing the rate of lamp degradation via electrode degradation and phosphor degradation. Maintaining uniform radiant intensities and powers is beneficial because it aids in ensuring a biological sample is exposed to sufficient electromagnetic radiation to provide a desired level of pathogen reduction. In addition, uniform radiant intensities and powers allow for the net amount of electromagnetic radiation delivered to a sample during a selected illumination period to be accurately measured and/or calculated. Exemplary illuminators of this aspect of the present invention achieve and maintain an optimal average lamp temperature for a given sample treatment application while at the same time minimizing the temperature gradients along the central longitudinal axes of one or more fluorescent lamps. In an exemplary embodiment useful for the treatment of blood and/or blood components, illuminators of the present invention maintain an average fluorescent lamp temperature of 40° C. and a temperature gradient across a central longitudinal axis which is less than 20° C., preferably for some applications less than 15° C., and more preferably for some applications less than 10° C.

An exemplary illuminator of the present invention having reduced temperature gradients comprises a tubular fluorescent light source and a differential cooling system. In this embodiment, the tubular light source extends along a central lamp axis and has a first end, a second end and a central region positioned between said first end and said second end.

The differential cooling system comprises a plenum chamber in fluid communication with the tubular light source and having a fluid intake vent for generating a primary flow into the plenum chamber, and a fluid distribution means positioned proximate to the light source. The fluid distribution means has a pattern of orifices for generating a distribution of secondary flows directed toward the surface of the light source. In an exemplary embodiment, the area, position, and shape of the orifices of the fluid distribution means, or any combination of these variables, are selected to provide a desired external surface temperature profile of the light source along the central lamp axis. In an exemplary embodiment useful for improving lamp longevity, the pattern of orifices in the fluid distribution means is selected such that the light source has a substantially uniform temperature along the central lamp axis.

Differentially cooling systems of the present invention operate via differential forced convection cooling. In this method, a distribution of secondary flows is directed toward the external surfaces of one or more light sources to provide cooling via convective heat transfer between the fluid and the external surfaces of the light sources undergoing cooling. Secondary flows useable for differential cooling in the present invention include turbulent flows, laminar flows or flows having both turbulent and laminar flow components. Secondary flows useable for differential cooling in the present invention can have any fluid flow direction which provides for effective convective heat transfer between the fluid and the surface undergoing cooling.

In the context of this invention, the phrase “distribution of secondary flows” refers to a plurality of discrete fluid flows, which are each individually directed toward a surface undergoing cooling. Each fluid flow in the distribution can be characterized by a respective mass flow rate, fluid flow direction and an area of thermal contact between the flow and the surface undergoing cooling. The cumulative convective cooling provided by the plurality of secondary flows establishes a selected temperature profile of the external surfaces of the light sources undergoing forced convection cooling. The distribution of fluid flows can be non-uniform and reflect a range of mass flow rates, fluid flow directions, thermal contact areas or any combination of these. Secondary flow directions useable in the present invention include fluid flow directions having a component which is oriented orthogonal to the surface undergoing cooling. Alternatively, fluid flows having non-orthogonal fluid flow components can be employed. In an exemplary embodiment, mass flow rates, directions of secondary flows and thermal contact areas are selected to achieve a desired temperature profile along the external surface of one or more light sources. For example, the distribution of secondary flows establishes and maintains a substantially uniform external surface temperature along a central longitudinal axis of one or more fluorescent lamps. The distribution of fluid flows of the present invention can reflect a plurality of flows along a defining axis, such as the central lamp axis of a tubular fluorescent lamp. Alternatively, the distribution of fluid flows can reflect a plurality of fluid flows distributed throughout a two dimensional area, such as a two dimensional area corresponding to the external surfaces of a plurality of fluorescent lamp light sources. Use of a plurality of secondary flows each having a selected mass flow rate, direction and thermal interaction area is beneficial because it allows precise control of the temperature of the external surfaces of light sources of the present invention at points along such defining axes or two dimensional areas.

In an exemplary embodiment, the fluid distribution means of the present invention comprises a fluid distribution plate positioned proximate to said light source. An exemplary fluid distribution plate of the present invention has an internal side proximate to the light source, an external side distal to the light source and a pattern of orifices extending through the internal and external sides. Patterns of orifice useable include patterns which are symmetrical about the central longitudinal axis of a fluorescent lamp or an axis oriented perpendicular to the central longitudinal axis. Alternatively, the present invention includes pattern of orifices which are asymmetrical with respect to the central longitudinal axis of a fluorescent lamp or an axis oriented perpendicular to the central longitudinal axis. Orifices useable in the present invention can have any shape, area and position on the fluid distribution plate which generates the appropriate distribution of secondary flows for a given light source geometry sample treatment, application or desired light source temperature profile. Orifice shapes useable in the present invention include, but are not limited to, round, obrotund, rectangular, square, diamond and trapezoidal. Orifice shape, area and position establish the magnitude of the secondary flows for a given primary mass flow rate. Further, orifice shapes, areas and positions establish which regions of the external surface of the light source undergo thermal energy transfer with a given secondary flow or plurality of secondary flows.

Optionally, illuminators of this aspect of the present invention further comprise additional light sources having differential cooling. In one embodiment, additional tubular fluorescent light sources are positioned along additional lamp axes oriented parallel to the central lamp axis and located in a common plane. In this embodiment, the plurality of light sources form a lamp bank providing a distribution of radiant intensities which is substantially spatially uniform. In another embodiment, additional tubular fluorescent light sources are positioned in a plurality of planes and, thus, form a plurality of lamp banks. In an exemplary embodiment, tubular fluorescent light sources occupy planes both above and below the biological sample undergoing treatment to provide high radiant intensities and powers. In another exemplary embodiment, each additional light source is in fluid communication with a plenum chamber and a fluid distribution means. In an exemplary embodiment, the area, position, and shape of the orifices of the fluid distribution means or any combination of these variables is selected to provide a desired external surface temperature profile of each additional light source along its corresponding additional lamp axis. Importantly, methods and devices providing differential cooling of the present invention avoid or reduce temperature gradients caused by thermal interaction between light sources in a multiple light source illuminator. In an exemplary embodiment, the differential cooling system of the present invention provides larger secondary flows to light sources positioned in the center of a plurality of parallel lamps distributed over a lamp bank plane than to light sources positioned at the ends of the lamp bank. Accordingly, the differential cooling system of the present invention is capable of maintaining substantially the same temperature of each light source in a multiple light source illuminator.

Selection of orifice shape, area and position in fluid distribution means of the present invention is determined by applying principles of the field of fluid mechanics. First, the cumulative area of the orifices in the fluid distribution means should be approximately equal to the area of the fluid intake vent to ensure establishing a stable distribution of secondary flows is established and to avoid the occurrence of back pressure against the intake vent. Second, larger orifice areas can be provided proximate to hotter areas of the surface undergoing cooling to achieve greater mass flow rates and corresponding greater transfer of thermal energy from these regions. Third, larger orifice areas can be provided to achieve a corresponding larger thermal interaction area between a selected secondary flow or plurality of secondary flows and a region of the external surface undergoing convective cooling. Fourth, the positions of orifices in the differential cooling system of present invention can be selected to take into account changes in the momentum of the primary flow as it flows through the plenum chamber. In an embodiment providing equivalent secondary flows proximate to the intake vent and at a selected distance from the intake fan, a first orifice having a larger area is positioned proximate to the intake vent and a second orifice having a smaller area is positioned a selected distance from the intake fan. Selection of the appropriate ratio of orifice areas provides approximately equal mass flow rates to the surface undergoing cooling due to the decrease in momentum experienced as the primary flow passes through the plenum chamber. This aspect of the invention is useful for providing substantially equivalent secondary flows to the cathode regions of a differentially cooled fluorescent lamp. Fifth, the fluid used for cooling can be selected on the basis of its heat-transfer coefficient. For example, a fluid can be chosen having a heat-transfer coefficient optimized for a particular differential cooling application. Sixth, the distance separating the fluid distribution means and the surface undergoing cooling can be selected to achieve a desired thermal contact area. Specifically, orifices of the present invention can be positioned closer to the light source external surface to provide a smaller thermal interaction area or orifices can be positioned farther away from the light source external surface to provide a larger thermal contact area. Finally, the selection of a combination of orifice shapes, areas and positions necessary to provide optimal differential cooling for a given light source or combination of light sources can be determined empirically or can be determined using fluid mechanical numerical models, such as the CF Design software package of Atgroup Software.

In another aspect, the present invention provides illuminators for pathogen or leukocyte reduction which generate radiant intensities and powers that are substantially constant as a function of time, particularly for a sample treatment time selected to achieve reduction of pathogen or leukocyte concentrations to a desired level. An exemplary illuminator having substantially constant radiant intensities and powers comprises one or more tubular fluorescent lamp or other light sources having external surface temperature profiles along a central longitudinal axis which do not vary substantially during illumination. Illuminators having substantially constant radiant intensities, powers or both are beneficial because they provide sample illumination conditions which are independent of variations in ambient conditions, such as ambient temperature, relative humidity and pressure. Additionally, illuminators having substantially constant radiant intensities, powers, or both, are beneficial because they provide pathogen or leukocyte reduction rates capable of being accurately calculated or quantified as a function of illumination time. This aspect of the present invention is especially useful for ensuring that a given illumination period is long enough to achieve an extent of pathogen reduction necessary for the safe use of a treated biological sample as a therapeutic agent.

The present invention provides an illuminator having closed loop feedback temperature control for maintaining substantially constant radiant intensities and powers during a selected sample illumination period. An exemplary illuminator having closed loop feedback temperature control comprises a light source, a cooling system, a temperature sensor and a temperature controller. The cooling system in one embodiment comprises a plenum chamber in fluid communication with the light source. An exemplary cooling system comprises a fluid intake vent, a fluid distribution means, and a selectively adjustable flow actuator for generating a primary flow having a selected mass flow rate into said plenum chamber. In this embodiment, the fluid distribution means generates at least one secondary flow for convectively cooling the external surface of the light source. In one embodiment, the temperature sensor is operably coupled to the light source in a manner such that it generates an output signal corresponding to the temperature of the external surface of the light source, for example the temperature of a detection area positioned the external surface of the light source. In the context of this description, “operably coupled” refers to a configuration of two or more device elements such that they are capable of being used in combination to achieve specific functions, operations, functional tasks or device capabilities/features in a particular device configuration. In one embodiment, the temperature sensor is optically coupled to the light source such that a portion of the light generated from the light source(s) impinges upon the temperature sensor, thereby allowing the temperature sensor to measure the temperature of the light source. Use of a temperature sensor optically coupled to the light source and positioned in a region proximate to the sample undergoing illumination is useful for some applications of the present methods. For example, the temperature sensor can be attached to a sample compartment, a vessel containing the sample, a sample support such as a shelf, drawer or base holding the vessel containing the sample or a sample agitator. Alternatively, the temperature sensor can be attached to the light source housing such as the walls of the light source housing, the reflective side of a fluid distribution plate, the light source itself, or a region of the plenum chamber in optical communication with the light source such as a region of the plenum chamber positioned behind an orifice or slot in a fluid distribution such that light from the light source passes through the orifice or slot and impinges on the temperature sensor. The present invention also includes embodiments having a plurality of temperature sensors operably coupled to a plurality of detection areas on a single light source or plurality of light sources.

The temperature controller in one embodiment is operably connected to the temperature sensor and the selectively adjustable flow actuator. In an exemplary embodiment, the temperature controller receives an output signal from the temperature sensor, compares the output signal to a pre-selected set point temperature, and adjusts the flow actuator to establish and maintain a substantially constant temperature of the detection area on the external surface of the light source. In some embodiments, the flow actuator is selectively adjusted to establish and maintain a substantially constant temperature equal to the pre-selected set point temperature.

In this aspect of the present invention, the temperature sensor directly measures the temperature of a detection area on the external surface of the light source, such as the temperature in a region which is representative of the temperature profile of the external surface, and generates a corresponding output signal. The temperature controller receives the output signals from the temperature sensor and compares the measured temperature to a pre-selected set-point temperature. In the event that the measured temperature and set-point temperature are not equal, the temperature controller adjusts the flow actuator to generate a primary flow necessary to establish a detection area temperature equal to the set point temperature. Specifically, the flow actuator is adjusted in a manner providing an increase or decrease in the primary fluid mass flow rate, which in turn increases or decreases the secondary fluid rates directed at the external surface of the light source. Accordingly, illuminators of this aspect of the present invention establish and maintain a detection area temperature equal to the set-point temperature for a selected sample treatment time, such as a sample treatment time selected over the range of about 5 to 30 minutes. This allows the radiant output of illuminators of the present invention to be accurately quantified and calibrated as a function of light source surface temperature. In addition, the temperature control provided by the methods of the present invention is useful for maintaining radiant intensities, radiant powers and a distribution of emission wavelengths at which photoreduction of pathogens in a biological sample is highly effective.

The pre-selected set point temperature of this aspect of the present invention can be selected to provide the optimal temperature for light source operation for a particular sample treatment application or illuminator function. In an exemplary embodiment, for example, a set point temperature is selected which provides the lowest current through a mercury vapor fluorescent lamp necessary to provide a desired radiant power and intensity. This set point temperature can be determined empirically or can be selected on the basis of the phosphor present in the fluorescent lamp. In an exemplary embodiment particularly useful for illuminators having one or more mercury vapor fluorescent lamps, the set point temperature is equal to about 40° C. Selection of a set point temperature establishing the lowest lamp current for a selected radiant power is beneficial because it maximizes the longevity of the fluorescent lamp. Alternatively, a set point temperature can be selected providing radiant output stability, net radiant output power, distribution of emission wavelengths or any combination of these which is optimized for a selected sample treatment application or biological sample undergoing treatment.

In an alternative embodiment, the present invention provides illuminators having closed loop feedback temperature control wherein periodic, direct in situ measurements of light intensities and/or radiant power are used to generate control signals that establish and maintain a light source temperature corresponding to desired irradiance conditions, such as radiant intensities and/or radiant powers that are substantially constant as a function of time. In an exemplary embodiment, an irradiance detector is provided which directly measures the light source radiant power and/or intensity as a function of time. Exemplary irradiance detectors measure the power and/or intensity of light having wavelengths capable of providing direct photoreduction of pathogens or photoinduced chemical reduction. Irradiance detectors of the present invention are any device, devices or device components capable of measuring the intensity and/or power of the light source and generating an output signals corresponding to the intensity and/or power, such as a photodiode, photo conductive detector or photomultiplier tube. In a exemplary embodiment, the irradiance detector is positioned proximate to the biological sample undergoing treatment, for example positioned in a sample compartment housing the biological sample. Positioning the irradiance detector proximate to the biological sample undergoing treatment is useful because provides a direct measurement of the irradiance delivered to the sample during treatment. However, the present invention also includes embodiments wherein the irradiance detector is positioned in a region spaced apart from the sample undergoing irradiance. For example, an irradiance detector can be positioned in optical communication with the side of a light source which is opposite to the sample undergoing treatment, such as the side of a light source facing the distribution surface. In these embodiments, it is necessary to correlate the radiant intensities and/or powers measured to the radiant intensities and/or powers exposed to the sample undergoing treatment by calibration means well known in the art.

Optionally, the methods and devices of this aspect of the present invention further include one or more optical filters positioned between the irradiance detector and the light source. Use of optical filters in the present invention is beneficial because it allows for selective detection of light having a selected wavelength range, such as light absorbed by a photosensitive compound present in the biological sample undergoing treatment. For example, filters are useful for avoiding the detection of infrared radiation which does not provide for direct photoreduction or photoinduced chemical reduction, but is detected with great sensitivity by the irradiance detector. In addition, optical filters can be used to avoid measurement of light originating from sources other than the illuminator light source, such as scattered room light and infrared radiation emitted by bodies in the chamber housing the sample.

The present invention also includes embodiments having more than one irradiance detector. Use of a plurality of irradiance detectors is beneficial because it provides the ability to monitor intensities corresponding to a plurality of different selected wavelength ranges. Redundant irradiance monitoring is useful because it provides an enhanced reliable measurement of the average irradiance delivered to a biological sample undergoing treatment. In addition, use of a plurality of detectors is beneficial because it provides the ability to monitor light intensities in a plurality of locations, which allows for characterization of the spatial distribution of light generated by the light source.

In one embodiment, the irradiance detector is operably coupled to a temperature controller and cooling system, such as those previously described in the context of illuminators employing closed loop feedback temperature control. In this embodiment, the irradiance detector directly measures the radiant intensity and/or power of light impinging on the detector as a function of time. Periodically, the irradiance detector generates output signals which correspond to detected light intensities or radiant powers over a selected light detection time interval. A temperature controller receives the output signals from the irradiance detector and compares the measured intensities and/or powers to a pre-selected irradiance set point, for example an irradiance set point corresponding to the maximum intensity and/or power of a fluorescent lamp for a selected, constant current passing through the lamp. In the event that the measured intensity and/or power and the irradiance set point are not equal, the temperature controller adjusts the cooling system in a manner necessary to achieve a light source temperature which establishes a radiant output providing the desired illumination conditions. Therefore, illuminators of this aspect of the present invention are capable of establishing and maintaining a pre-selected radiant output, such as a maximum radiant output for given current and/or power consumption conditions of the light source.

In one embodiment, illuminators having temperature feedback control further comprise additional tubular light sources, such as additional tubular light sources oriented along additional lamp axes parallel to the central lamp axis. In one embodiment, each light source in the illuminator is provided with a separate temperature sensor or irradiance detector. In this embodiment, the temperature controller averages output signals from the additional temperature sensors, and/or irradiance detector and adjusts the flow actuator to establish and maintain an average temperature equal to the set point temperature. Alternatively, the present invention includes embodiments wherein an illuminator having a plurality of light sources is temperature controlled by a plurality of temperature feedback control systems or irradiance feedback control systems.

The temperature control methods and devices of the present invention are highly versatile and can be used with light sources other than fluorescent lamps. For example, the methods of the present invention are well suited for controlling the temperature and radiant output of light emitting diode (LED) light sources, such as a plurality of LEDs configured in a LED array. LED light sources provide excellent wavelength specificity and high intensity. In addition, LED light sources have long lifetimes and are robust and inexpensive. Therefore, these light sources are particularly well suited for a wide variety of blood processing applications requiring high intensity light of a specific wavelength range. The methods and devices of the present invention improve the performance of LED light sources used in the treatment biological samples, such as blood and components of blood.

In an exemplary embodiment, the present invention provides closed loop feedback temperature control methods and devices for establishing and maintaining a constant temperature of a LED array comprising a plurality of LEDs. In one embodiment, one or more temperature sensors are operably coupled to a heat sink in thermal contact with a LED array. In one embodiment, the heat sink is located on the side of the LED array opposite of the sample undergoing treatment and can be cooled by forced convective cooling methods providing a selectively adjustable cooling rate. In an exemplary embodiment, output signals corresponding to the temperatures of LEDs in the LED array are generated by the temperature sensors and serve as the basis of control signals that establish and maintain a pre-selected set point temperature. In an exemplary embodiment, output signals are sent to a temperature controller which controls the forced convection cooling conditions experienced by the heat sink, for example by increasing or decreasing the mass flow rate of a cooling fluid flowing past the heat sink. For example, the temperature controller establishes a heat sink temperature providing the longest lifetime of LED light sources in the array, maximum irradiance for a given LED current draw, the greatest radiant output stability, or any combination of these variables. In an alternative embodiment, closed loop feedback temperature control is achieved using one or more irradiance detectors in optical communication with the LED array light source. The present methods of differential cooling can also be applied to controlling temperature gradients and radiant outputs of LED array light sources.

Illuminators of the present invention provide for treatment of biological samples with light having wavelengths selected over the range of about 270 nm to about 800 nm, preferably for some applications over the range of about 340 nm to about 650 nm. Exemplary illuminators of the present invention generate light having a distribution of wavelengths in the visible range, ultraviolet range or both. The optimal wavelength of light for achieving photoinduced chemical reduction of pathogens in biological samples, however, depends on the identities of photosensitive compounds used for reduction. Typically, the optimal wavelength for illumination will reflect the absorption maxima of the photosensitive compounds added to the biological sample.

The differential cooling methods, temperature control techniques and irradiance control methods of the present invention are especially well suited for applications involving high intensity illumination, such as illumination with radiant powers greater than 10 mW cm⁻². Exemplary ultraviolet light illuminators of the present invention provide for the treatment of biological samples with light having a power per square centimeter selected over the range of about 10 mW cm⁻² to about 75 mW cm⁻² and exemplary visible light illuminators of the present invention provide for the treatment of biological samples with light having a power per square centimeter selected over the range of about 10 mW cm⁻² to about 120 mW cm⁻². Use of lower radiant power per square centimeter is useful for some applications to avoid damage to cellular components and noncellular components present in the sample which are useful as therapeutic agents. For example, high radiant powers can result in elevation of the temperature of the biological sample to temperatures high enough to damage cellular and noncellular components or can result in direct photoinduced decomposition of materials, such as proteins, present in the sample.

Biological samples which can be treated by the methods and devices of the present invention include any material which is adequately transmissive to electromagnetic radiation of a selected wavelength range, particularly visible light, ultraviolet light or both. Biological samples treatable by the devices and methods of the present invention include liquid samples, solid samples and samples comprising colloidal suspensions. Exemplary materials include, but are not limited to, whole blood, blood components, aqueous compositions containing compounds derived from blood samples, and tissue samples. Examples of blood components include but are not limited to erythrocytes, leukocytes, thrombocytes, and plasma. Examples of compounds derived from blood samples include, but are not limited to, biologically active proteins such as factor III, Von Willebrand factor, factor IX, factor X, factor XI, Hageman factor, prothrombin, anti-thrombin III, fibronectin, plasminogen, plasma protein fraction, immune serum globulin, modified immune globulin, albumin, plasma growth hormone, somatomedin, plasminogen, streptokinase complex, ceruloplasmin, transferrin, haptoglobin, antitrypsin and prekallikrein.

Biological samples treatable by the present methods and devices include static samples held in a container which is at least partially transmissive to electromagnetic radiation. Containers for static samples can be rigid or made of a flexible material and include but are not limited to transmissive bags, boxes, tubes, cuvettes and troughs. Alternatively, the present methods and devices can be used to treat flowing samples, such as liquid samples or solid samples suspended in a liquid. In these embodiments, the sample undergoing irradiation is flowed past the illuminator in a transmissive tube or other transmissive illumination reactor. Optionally, biological samples are agitated while being treated by the methods and devices of the present invention to ensure uniform exposure of all portions of the sample to electromagnetic radiation. Containers and flow reactors useful for the treatment of biological samples can have spatially uniform transmission properties, particularly in the region exposed to electromagnetic radiation.

Illuminators of the present invention are stand-alone-units or are integral components in a larger apparatus. For example, illuminators of the present invention can be incorporated into blood processing apparatuses known in the art for separating or treating whole blood or blood components withdrawn from or administered to a patient or donor. Illuminators of the present invention can be incorporated into apheresis systems such as the COBE Spectra® or TRIMA® apheresis systems, available from Gambro BCT, Lakewood, Colo., USA. Illuminators of the present invention can be positioned proximate to and downstream of the point wherein blood is withdrawn from a patient or donor, proximate to the point wherein a blood component or product is transfused into a patient or at any point before or after separation of blood components.

The present invention also includes methods, devices and device components for treating surfaces to reduce the biological activity of microorganisms which can be present thereon. In this aspect of the invention, the surface is exposed to electromagnetic radiation generated by an illuminator of the present invention. The biological activity of microorganisms present at or within the surface is either directly reduced by photoreduction or indirectly reduced by photoinduced chemical reduction.

In another aspect, the present invention provides methods for differentially cooling the external surface of a light source for treating a biological sample, comprising the steps of: (1) generating a primary flow in a plenum chamber in fluid communication with a light source which extends along a central axis; and (2) passing at least a portion of the primary flow through a fluid distribution means having a pattern of orifices positioned proximate to the light source, thereby generating a distribution of secondary flows directed toward the external surface of the light source. In an exemplary method, the area, position and shape of the orifices, or any combination of these parameters, is selected such that the surface of the light source has a desired temperature profile along said central axis.

In another aspect, the present invention provide methods for establishing and maintaining a substantially constant external surface temperature of a light source used for treating a biological sample, comprising the steps of: (1) measuring the temperature of an external surface of a light source, said light source having a cooling system comprising: a plenum chamber having a fluid intake vent, a fluid distribution means in fluid communication with the light source, and a selectively adjustable flow actuator for generating a primary flow having a selected mass flow rate into said plenum chamber, wherein the fluid distribution means generates at least one secondary flow directed toward the external surface of the light source for convectively cooling the external surface of said light source; (2) comparing the measured temperature to a pre-selected set point temperature; and (3) adjusting the flow actuator to provide a primary fluid mass flow rate which establishes and maintains a substantially constant temperature of the external surface of the light source equal to the pre-selected set point temperature.

The invention is further illustrated by the following description, examples, drawings and claims.

### BRIEF DESCRIPTION OF THE DRAWINGS

FIGS. 1A and 1B are schematic drawings showing views of an illuminator of the present invention having differential cooling. FIG. 1A illustrates a horizontal side view and FIG. 1B illustrates an end view.

FIGS. 2A and 2B are schematic drawings showing top plan views of exemplary fluid distribution plates useable in the present invention. The fluid distribution plate shown in FIG. 2A has a pattern of orifices which is asymmetrical with respect to the fluid distribution plate axis and the fluid distribution plate shown in FIG. 2B has a pattern of orifices which is symmetrical with respect to the fluid distribution plate axis.

FIG. 3 provides a flow chart illustrating a closed loop feedback temperature control method useful for maintaining substantially constant radiant intensities and radiant powers of an illuminator of the present invention.

FIG. 4. is a schematic drawing showing an exemplary ultraviolet light illuminator of the present invention having first and second differentially cooled light sources.

FIG. 5. is a schematic drawing of a first differentially cooled light source having 6 tubular fluorescent lamps, which is a component of an exemplary ultraviolet light illuminator of the present invention.

FIG. 6. is a schematic of a first differentially cooled light source of an exemplary ultraviolet light illuminator of the present invention having the fluorescent lamps removed from view.

FIG. 7 shows a plot of lamp temperatures (top plots) and fan speed (bottom plots) verses time observed for an exemplary ultraviolet light illuminator having closed loop feedback temperature control.

FIG. 8 is a schematic drawing showing an exemplary visible light illuminator of the present invention having first and second differentially cooled light sources.

FIG. 9 is a schematic drawing of a first differentially cooled light source having 7 U-shaped fluorescent lamps, which is a component of an exemplary visible light illuminator of the present invention.

FIG. 10 is a schematic of a first differentially cooled light source of an exemplary visible light illuminator of the present invention having the fluorescent lamps removed from view.

### DETAILED DESCRIPTION OF THE INVENTION

Referring to the drawings, like numerals indicate like elements and the same number appearing in more than one drawing refers to the same element. In addition, hereinafter, the following definitions apply:

“Differential cooling” refers the process of cooling a surface or plurality of surfaces in a manner such that the rate of heat transfer from the surface or a plurality of surfaces undergoing cooling varies along a defining axis or within a two dimensional area. Differential cooling systems of the present invention provide selectable control of the thermal energy transfer rates across a surface or plurality of surfaces undergoing cooling. Differential cooling systems of the present invention utilize a distribution of secondary flows along a defining axis or in a two dimensional area having selected mass flow rates, directions and thermal contact areas, which are directed onto regions of the surfaces of one or more light sources undergoing cooling. In an exemplary embodiment, an illuminator of the present invention is differentially cooled in a manner establishing and maintaining a selected temperature profile along the surface of one or more light sources, such as a substantially uniform temperature profile. Exemplary differential cooling systems establish larger heat transfer rates in higher energy-producing regions of a light source, such as the regions proximate to a cathode, compared to lower energy-producing regions of a light source, such as the center of a fluorescent lamp.

“Fluid” refers to any material which is capable of conforming to the shape of the container in which it is held. Fluid flows are used in the present invention to provide convective cooling of one or more surfaces of light sources. Fluids useable for providing convective cooling include, but are not limited to, liquids, gases and colloidal mixtures. Exemplary fluids include air, N₂, O₂, He, Ar, and CO₂.

“Intake vent” and “fluid intake vent” are used synonymously and refer to an opening or plurality of openings for passing a fluid flow. Intake vents of the present invention can have any shape including, but not limited to, rectangular, circular, oval, trapezoidal, square and triangular. Intake vents comprise a single opening of a selected shape or comprise a plurality of openings having different shapes. In an exemplary embodiment, an intake vent of the present invention is an opening in a plenum chamber whereby a primary fluid flow passes from outside the plenum chamber into the plenum chamber. Optionally, intake vents of the present invention further comprise a flow diffuser, such as a diffuser plate. In one embodiment, diffuser plates of the present invention have a porosity selected over the range of about 50% to about 95%.

“Light source” refers to any device or material capable of generating electromagnetic radiation or a plurality of devices or materials capable of generating electromagnetic radiation. Exemplary light sources of the present invention comprise one or more fluorescent lamps. Light sources of the present invention are capable of providing electromagnetic radiation to a biological sample undergoing treatment, particularly light having a selected distribution of wavelengths in the visible region, ultraviolet region or both. Exemplary light sources useable in the present invention include, but are not limited to, mercury vapor fluorescent lamps, cold cathode fluorescent lamps, excimer lamps, light emitting diodes (LEDs), arrays of light emitting diodes, arc discharge lamps and tungsten-filament lamps.

“In fluid communication” refers to materials, devices and device components that are in contact with a fluid such as a primary flow, secondary flow or both. Materials, devices and device components in fluid communication can be characterized as upstream or downstream of each other with respect to the net direction of fluid flow.

“Plenum” refers to an space wherein the pressure of a fluid is higher within the space relative to the pressure of fluid at some point outside the space. An exemplary plenum useful in the present invention is a plenum chamber comprising an enclosed space wherein the pressure of a fluid is higher inside the chamber relative to the pressure of fluid outside the chamber. The higher pressure region inside the plenum chamber can be established by a flow of fluid through an opening into the plenum chamber, such as an intake vent. Plenum chambers of the present invention are capable of generating a plurality of secondary flows, having selected mass flow rates and directions, which are directed outside the chamber toward one or more surfaces undergoing differential cooling.

“A substantially uniform temperature profile” refers to a distribution of temperatures along a selected defining axis or over a two dimensional area which is characterized by a substantially constant temperature. Exemplary illuminators of the present invention comprise light sources having external surfaces characterized by substantially uniform temperature profiles. The term substantially uniform temperature profile is intended to include some extent of temperature gradients along a selected defining axis or over a two dimensional area. In an exemplary embodiment, a light source having an external surface with a substantially uniform temperature profile is characterized by a temperature gradient less than 20° C. along a central longitudinal axis. In an exemplary embodiment, a light source having an external surface with a substantially uniform temperature profile is characterized by a temperature gradient less than 15° C. along a central longitudinal axis. In another embodiment providing improved lamp longevity, a light source having an external surface with a substantially uniform temperature profile is characterized by a temperature gradient less than 10° C. along a central longitudinal axis.

“Central lamp axis” refers to an axis along which a light source extends. In an exemplary embodiment, the central lamp axis is an axis along which a linear tubular lamp extends. In another embodiment, the central lamp axis is an axis which extends along straight portions of a U-shaped tubular lamp and passes through the center of the curved lamp region. In yet another embodiment, the central lamp axis is an axis which passes through the center of a circular light source, such as a circular array of LEDs. Exemplary illuminators of the present invention have light sources characterized by a substantially uniform profile along the central lamp axis.

“Fluid distribution means” refers to a device or device component capable of generating a plurality of secondary flows from a primary flow. Fluid distribution means are capable of generating secondary flows comprising a number of fluid flows distributed over a defining axis or defining area. Exemplary fluid distribution means of the present invention are capable of generating secondary flows having selected directions, mass flow rates and thermal contact areas with a surface undergoing differential cooling. Exemplary fluid flow distribution means of the present include, but are not limited to, a fluid distribution plate having a pattern or patterns of orifices, a series of flow guides or flow channels or a combination of these. Fluid distribution plates of the present invention have flat regions, curved regions or a combination of flat and curved regions.

“Primary flow” refers to a flow of fluid which is capable of generating one or more secondary flows. In an exemplary embodiment, a primary flow is directed into a plenum in a manner resulting in the formation of one or more secondary flows directed at a surface undergoing cooling, such as the external surface of a light source. Primary flows of the present invention can be characterized in terms of their mass flow rate, direction fluid composition, Reynolds number or any combination of these variables. Primary flows of the present invention can be generated by means well known in the art of fluid mechanics including, but not limited, using fans, fluid actuators and fluid pumps.

“Secondary flow” refers to a flow of fluid or plurality of flows of fluid which is generated from a primary flow. Secondary flows can be generated by establishing a primary flow into a plenum chamber having a fluid distribution means. In exemplary methods of the present invention, secondary flows are used to control the temperature of a light source and/or the temperature distribution of a light source along one or more axis or along one or more areas. Secondary flows of the present invention can be characterized in terms of their mass flow rate, direction, position, fluid composition, Reynolds number, thermal interaction area or any combination of these variables.

“Pathogenic contaminants” and “pathogens” refer to viruses, bacteria, bacteriophages, fungi, protozoa, blood-transmitted parasites. Exemplary viruses include acquired immunodeficiency (HIV) virus, hepatitis A, B, C and G viruses, sindbis virus, cytomegalovirus, vesicular stomatitis virus, herpes simplex viruses, human T-lymphotropic retroviruses, HTLV-III, lymphadenopathy virus LAV/IDAV, parvovirus, transfussion (TT) virus, Epstein-Barr virus, West Nile virus and others known to the art. Exemplary bacteriophages include but are not limited to ΦX174, Φ6, λ, R17, T4 and T2. Exemplary bacteria include P. aeruginosa, S. aureus, S. epidernis, L. monocytogenes, E. coli, K pneumonia and S. marcescens. Exemplary parasites include malaria, babesia and trypanosome.

“Biologically active” refers to the capability of a composition, material, microorganism, or pathogen to effect a change in a living organism or component thereof.

“Pathogen reduction” refers to processes which partially or totally prevent pathogens from reproducing. Pathogen reduction can occur by directly killing pathogens, interfering with their ability to reproduce, or a combination of these processes. In an exemplary embodiment, the methods and devices of the present invention are capable of treating a biological sample such that it is safe for use as a therapeutic agent.

“Mass flow rate” refers to the rate at which an amount of mass of a fluid or mixture of fluids passes through a plane. Mass flow rate can be expressed in terms of flow velocity via the expression:
mass flow rate=(ρ)(V)(A), (I)
where ρ is the density of the fluid or mixture of fluids (g cm⁻³), V is the linear flow velocity (m s⁻¹) and A is the area through which the fluid passes (cm²).

“Blood,” “blood product” and “blood component” as used herein include whole blood samples, blood components and blood products which can be derived from whole blood. Cellular blood components treatable by the present methods and devices include, but are not limited to erythrocytes, leukocytes, esinophils, monocytes, lymphocytes, granulacytes, basophils, plasma, and blood stems cells. Non-cellular blood components include blood proteins isolated from blood samples including, but not limited to, factor III, Von Willebrand factor, factor IX, factor X, factor XI, Hageman factor, prothrombin, anti-thrombin II, fibronectin, plasminogen, plasma protein fraction, immune serum globulin, modified immune globulin, albumin, plasma growth hormone, somatomedin, plasminogen, streptokinase complex, ceruloplasmin, transferrin, haptoglobin, antitrypsin and prekallikrein.

“Flux of photons” or “photon flux” refers to the number of photons of light passing a defining area at a given time. Typically, photon flux is defined in units of: (number of photons) cm⁻² s⁻¹.

“Parallel surfaces” refers to a geometry in which two surfaces are equidistant from each other at all points and have the same direction or curvature. The term parallel is intended to include some deviation from absolute parallelism, preferably for some applications deviations less than 10 degrees.

“External surface” refers to a surface or plurality of surfaces of a device or device component, which is in contact with the ambient surroundings. External surfaces are capable of being cooled by forced convection methods, wherein a fluid flow thermally interacts with the external surface. Light sources of exemplary illuminators of the present invention have external surfaces which are cooled via forced convection.

“Closed loop feedback temperature control” refers to methods and devices of temperature control employing positive or negative temperature feedback loops. In an exemplary embodiment, the temperature of one or more light source surfaces, radiant power and/or radiant intensity is measured in real time and serves the basis of control signals for controlling light source cooling conditions. In an exemplary embodiment, light source temperatures or radiant powers are periodically measured and compared to a selected set-point temperature or radiant power. The difference between the observed temperature or radiant power and the set point serves the basis for adjustments to convective cooling conditions, which establish and maintain a light source temperature resulting in optimal light source longevity. Alternatively, the difference between the observed temperature or radiant power and the set point can serve the basis for adjustments to convective cooling conditions resulting in optimal irradiance conditions, such as optimal radiant stability, wave length distribution and/or radiant power, for a given application.

“Selectively adjustable flow actuator” refers to a device or device component which is capable of generating a fluid flow having a selectively adjustable mass flow rate. Selectively adjustable flow actuators of the present invention are capable of adjustment in a manner necessary to establish and maintain a constant temperature of a detection region on the external surfaces of one or more light sources or to establish and maintain selected irradiance conditions, such as radiant intensity or power. Selectively adjustable flow actuators useable in the present invention include, but are not limited to, fans having a selectively adjustable fan speed and pumps having a selectively adjustable pumping rate.

“Thermal interaction area” refers to a characteristic of a flow, such as a secondary flow, used in forced convection cooling of a surface. The thermal interaction area between a flow and a surface undergoing cooling is the region of the surface that transfers thermal energy to the flow upon interaction of the flow with the surface. Selection of a distribution of secondary flows, each having a selected thermal interaction area, is useful in the present invention in establishing a selected temperature profile across a surface undergoing forced convection cooling.

“Obrotund” refers to a two-dimensional shape characterized by a circle having first and second opposite-facing flattened ends.

“Substantially constant temperature” refers to the temperature of a material, detection area or device component exhibiting deviations from the average temperature or selected temperature over a useful time period that are not significant for a desired application or process. In one embodiment, a substantially constant temperature refers to a temperature of a material, detection area or device component exhibiting deviations from the average temperature or a selected temperature equal to or less than about 0.5 degrees Celsius over a time period equal to about 9 minutes. In another embodiment, a substantially constant temperature refers to the temperature of a material, detection area or device component exhibiting deviations from the average temperature or a selected temperature equal to or less than about 0.1 degrees Celsius over a time period equal to about 9 minutes.

“Operably connected” and “operably coupled” are used synonymously in the present description and refer to a configuration of two or more device elements such that they can be used in combination to achieve specific functions, operations, functional tasks or device capabilities/features in a particular device configuration. Operably connected device elements can be optically coupled, electronically coupled, electrically coupled, magnetically coupled or any combination of these. Operably connected device elements can be in fluid communication, in one way communication, in two way communication or any combination of these device configurations. Operably coupling device elements is used in the present invention to provide devices and device configurations having a desired functionality, such as an illuminator capable of providing substantially constant radiant intensities, radiant powers and/or light source temperatures. In one embodiment of the present invention, a temperature sensor is operably coupled to a one or more light sources, preferably optically coupled for some applications, in a manner such that the temperature sensor is capable of providing measurements of the temperature of the light source. In one embodiment providing an illuminator for treating a biological sample, a cooling system of the present invention operably connected to a light source, preferably in fluid communication for some applications, such that the cooling system is capable of adjusting the temperature of the light source. In one aspect of the invention, a cooling system of the present invention is operably coupled to a light source such that is capable of cooling the light source via convective cooling, for example by providing a secondary flow directed at an external surface of a light source. In one embodiment providing a cooling system of the present invention, a selectively adjustable flow actuator is operably connected to a fluid intake vent of a plenum chamber, preferably in fluid communication for some applications, in manner capable of generating a primary flow into the plenum chamber.

“Optically coupled” and “optically coupled” are used synonymously in the present description and refer to a configuration of two or more device elements wherein photons of light a capable of propagating from one element to another element. Device elements can be optically coupled using a variety of device components including, but not limited to, wave guides, fiber optic elements, reflectors, filters, prisms, lenses, gratings and any combination of these device components.

In the following description, numerous specific details of the devices, device components and methods of the present invention are set forth in order to provide a thorough explanation of the precise nature of the invention. It will be apparent, however, to those of skill in the art that the invention can be practiced without these specific details.

This invention provides methods, devices and device components for treating biological samples with electromagnetic radiation. In particular, the present invention provides illuminators which provide spatially uniform and substantially constant radiant intensities, which are especially useful for pathogen reduction in biological samples.

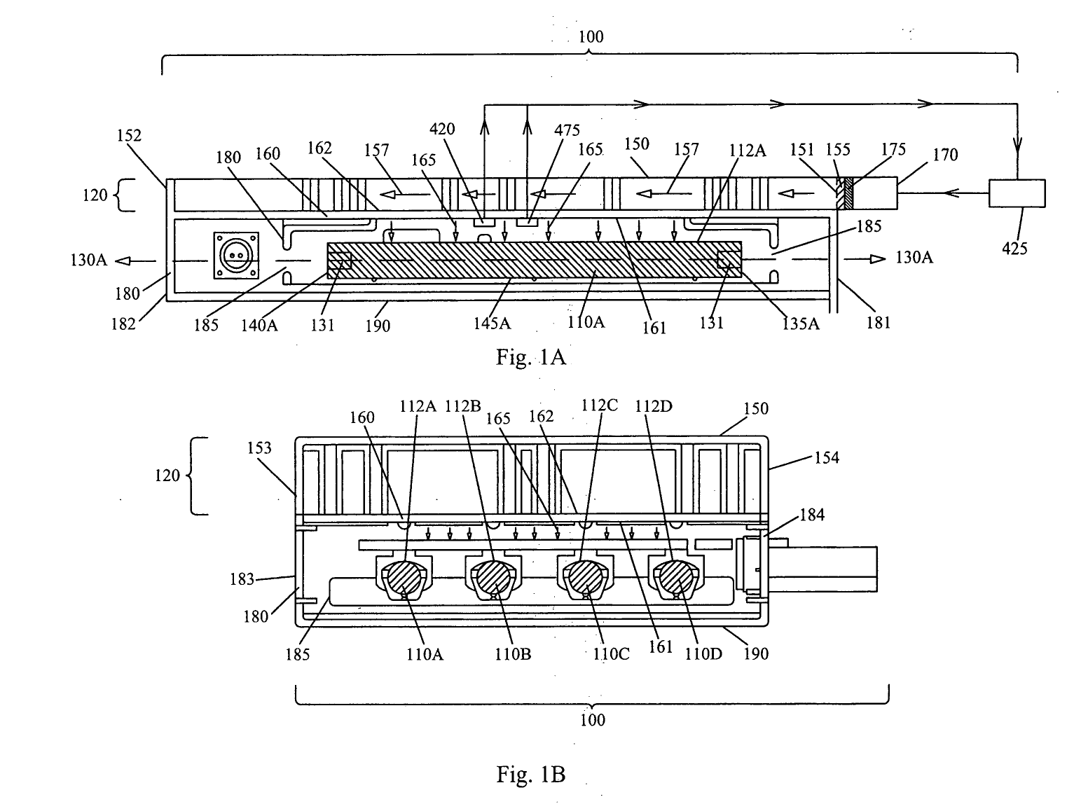

FIGS. 1A and 1B schematically illustrate side views of an exemplary embodiment of an illuminator of the present invention having differential cooling. FIG. 1A illustrates a horizontal side view and FIG. 1B illustrates an end view. The illustrated illuminator 100 comprises tubular light sources 110A, 110B, 110C and 110D having external surfaces (see FIG. 1B, 112A, 112B, 112C and 112D) in fluid communication with a differential cooling system 120. Tubular light sources 110A, 110B, 110C and 110D extend along central axes 130A (FIG. 1A) and have first ends 135A (FIG. 1A), second ends 140A (FIG. 1A) and center regions 145A (FIG. 1A) (central axes, first ends, second ends and center regions corresponding to light sources 110B, 110C and 110D are not shown in FIGS. 1A and 1B due to the perspectives provided by horizontal side and end views), respectively. In the exemplary embodiment illustrated in FIGS. 1A and 1B, tubular light sources 110A, 110B, 110C and 110D are each mercury vapor fluorescent lamps having cathodes 131 (FIG. 1A) positioned at their first and second ends. In this embodiment, light sources 110A, 110B, 110C and 110D are arranged such that their central axes are parallel.

Referring to FIG. 1A, differential cooling system 120 comprises a plenum chamber 150 in fluid communication with the light sources 110A, 110B, 110C and 110D and having sides 151 (FIG. 1A), 152 (FIG. 1A), 153 (FIG. 1B) and 154 (FIG. 1B). Plenum chamber 150 also has a fluid intake vent 155 for generating a primary flow (schematically depicted by arrows 157) in the plenum chamber 150 and a fluid distribution means 160 positioned proximate to the light source and having a pattern of orifices (not shown in FIGS. 1A and 1B). In the embodiment illustrated in FIG. 1, intake vent 155 is located on plenum chamber side 151. Alternatively, intake vent is positioned on any of the other plenum chamber sides 152, 153 and 154. Selection of the position of the intake vent 155 establishes the direction of primary flow 157 in plenum chamber 150. The present invention also includes embodiments wherein plenum chamber 150 is equipped with a plurality of intake vents.

Fluid distribution means 160 has an external side 161, positioned proximate to the light sources 110A, 110B, 110C and 110D, and an internal side 162, positioned distal to the light sources 110A, 110B, 110C and 110D. The fluid distribution means 160 is arranged such that it is capable of generating a distribution of secondary flows (schematically depicted by arrows 165) from primary flow 157, which are directed toward the external surfaces 112A, 112B, 112C and 112D of light sources 110A, 110B, 110C and 110D. The distribution of secondary flows 165 establishes the external surface temperature profiles of light sources 110A, 110B, 110C and 110D, particularly along the central axes corresponding to each light source. Alternatively, fluid distribution means 160 of the present invention comprises one or more flow guides (not shown in FIGS. 1A and 1B) which provide a means of establishing one or more selected secondary flows having selected mass flow rates and directions. An advantage of using flow guides, such as channels, tubes and ramps, for conducting the direction of fluid flow, in the present invention is that they provides more control over the secondary flow directions. Fluid flow directions in the present invention can be selected to provide a desired thermal interaction area on the surface undergoing cooling.

The external side 161 of fluid distribution means 160 is optionally reflective in a manner capable of directing light from light sources 110A, 110B, 110C and 110D away from plenum chamber 150 and toward a biological sample undergoing treatment. Exemplary fluid distribution means of the present invention have highly reflective external sides for substantially reflecting light generated by the light source, such as those having a reflectivity greater than 90%. Fluid flow distribution surfaces of the present invention are flat or curved. Use of a fluid distribution means that is highly reflective and curved is desirable for focusing light emitted from the light source onto a selected illumination area, such as a biological sample container or flowing illumination reactor.

The exemplary illuminator of the present invention optionally includes at least one flow actuator 170 (FIG. 1A) operably coupled to the intake vent 155 for generating the primary flow 157 in the plenum chamber 150. Use of a flow actuator 170, such as a fan or fluid pump, is useful in some embodiments because it is capable of establishing a substantially constant mass flow rate of the primary flow in the plenum chamber, such as a selectively adjustable constant mass flow rate. Flow conditions into the plenum chamber can be established by means known in the art of fluid mechanics. Further, an exemplary illuminator of the present invention optionally includes at least one flow diffuser 175 operably coupled to the intake vent 155. Incorporation of a flow diffuser 175, such as a plate having a plurality of orifices, into the illuminator 100 is beneficial because it provides for more spatially uniform primary flow conditions in the plenum chamber 150, which are useful for generating an appropriate distribution of secondary flows for a given light source geometry or differential cooling application. Further, use of a flow diffuser 175 provides for a primary flow which is uniformly distributed to all areas of the flow distribution surface.

As illustrated in FIGS. 1A and 1B, light sources 110A, 110B, 110C and 110D are enclosed in a light source housing 180 having sides 181,182,183 and 184. In the exemplary embodiment illustrated in FIGS. 1A and 1B, light source housing 180 has one or more exhaust vents 185 positioned in fluid communication with the fluid distribution means 160. Exhaust vents 185 are positioned on any of light source housing sides 181 (FIG. 1A), 182 (FIG. 1A), 183 (FIG. 1B) and 184 (FIG. 1B) and are capable of conducting secondary flows 165 out of illuminator 100. Optionally, one or more additional flow actuators (not shown in FIGS. 1A and 1B) are operably coupled to exhaust vents 185 to provide efficient transfer of secondary fluid flows out of illuminator 100. Optionally, light source housing 180 is equipped with transmissive plate 190, which can be used to physically separate light sources 110A, 110B, 110C and 110D from a biological sample undergoing treatment.

Light sources 110A, 110B, 110C and 110D of illuminator 100 are cooled by differential forced convection. In this method, flow actuator 170 generates a primary flow 157 having a selected mass flow rate through intake vent 155 into plenum chamber 150. Primary flow 157 can have a component which flows in a direction substantially parallel to the central axis 130A, substantially orthogonal to the central axis 130A or some angular orientation which is between these flow orientations. Primary fluid flow 157 establishes a higher pressure inside the plenum chamber 150 than in light source housing 180. The pressure differential formed results in the generation of secondary flows 165 into light source housing 180. Fluid distribution means 160 is capable of establishing a distribution of secondary flows 165 from primary fluid flow 157, which are directed to external surfaces 112A, 112B, 112C and 112D of light sources 110A, 110B, 110C and 110D (FIG. 1B). Specifically, fluid distribution means 160 establishes a plurality of discrete fluid flows, each having a selected mass flow rate, direction and thermal interaction area with respect to external surfaces 112A, 112B, 112C and 112D.

Interaction of secondary flows 165 and external surfaces 112A, 112B, 112C and 112D results in convective thermal energy transfer wherein energy from light sources 110A, 110B, 110C and 110D is transferred to secondary flows 165. The position, mass flow rate and direction of the secondary flows 165 established by fluid distribution means 160 provide the thermal transfer rates and thermal contact areas between the secondary flows and external surfaces 112A, 112B, 112C and 112D necessary to provide desired temperature profiles along the central axes of each light source. After thermal interaction with external surfaces 112A, 112B, 112C and 112D, secondary flows are conducted out of light source housing 180 via exhaust vents 185. This results in a net transfer of heat away from the illuminator 110. Optionally, exhaust vents 185 are positioned in a manner such that fluid exiting illuminator 100 is not drawn back into plenum chamber 150. This can be accomplished by positioning intake vent 155 on a different side of illuminator 100 than exhaust vent 185. Alternatively, one or more flow partitions (not shown in FIGS. 1A and 1B) are positioned between the intake vent 155 and exhaust vents 185 to prevent fluid exiting the illuminator from being drawn back into the plenum chamber.

In an exemplary illuminator of the present invention, the distribution of secondary flows is selected such that light sources 110A, 110B, 110C and 110D exhibit substantially uniform temperature profiles along their respective central axis. For illuminators having mercury vapor fluorescent lamp light sources, substantially uniform temperature profiles are accomplished by providing a distribution of secondary flows having greater mass flow rates of the secondary flows directed to the ends of the lamps proximate to cathodes than to the mass flow rates of the secondary flows directed to the center regions.

Any pattern of orifices capable of generating the appropriate distribution of secondary flows for a given light source geometry or desired temperature profile can be used in the present invention. Patterns of orifices useable in the present invention include patterns which are symmetrical or asymmetrical with respect to the central longitudinal axis of a fluorescent lamp or other defining axis. Orifices useable in the present invention can have any shape, area and position on the distribution surface, which is capable of generating an appropriate distribution of secondary flows for a given light source geometry, application or desired light source temperature profile.

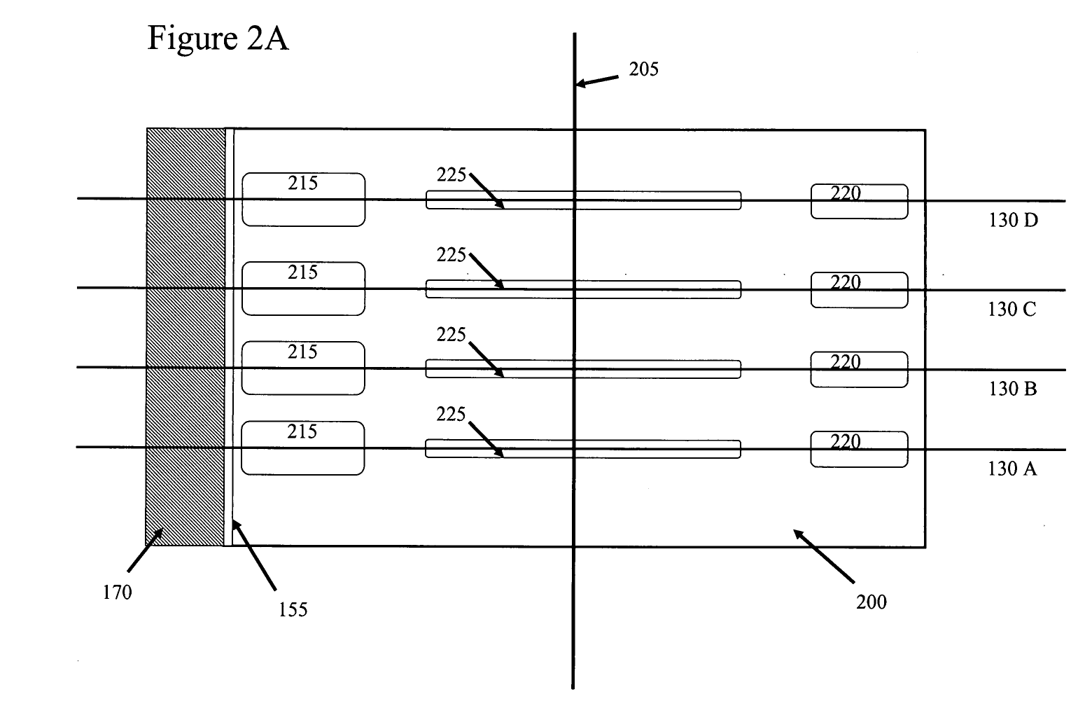

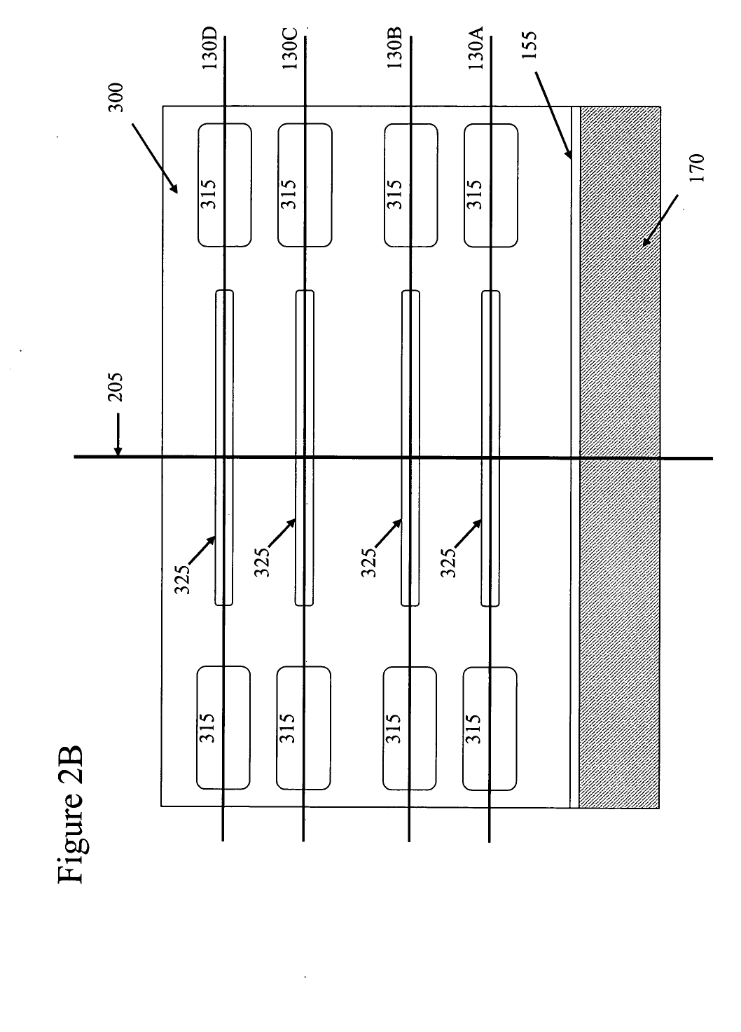

FIG. 2A shows a top plan view of an exemplary fluid distribution means comprising a fluid distribution plate 200, particularly useful for illuminators having light sources comprising mercury vapor fluorescent lamps with high energy producing cathodes. Fluid distribution plate 200 is characterized by a pattern of orifices which is asymmetrical with respect to the fluid distribution plate axis 205. The asymmetrical pattern of orifices is especially useful for establishing a substantially uniform temperature profile along the external surfaces of light sources 110A, 110B, 110C and 110D (FIG. 1B) in an illuminator configuration having a flow actuator 170 and fluid intake vent 155 positioned in a manner capable of generating a primary flow having a component orthogonal to fluid distribution plate axis 205. In the exemplary embodiment shown in FIG. 2A, the pattern of orifices includes large orifices 215, small orifices 220 and slits 225 positioned along the central axes of light sources 110A, 110B, 110C and 110D (FIG. 1B). In one embodiment, light sources (not shown in FIG. 2A) are positioned along central axes 130A, 130B, 130C and 130D indicated in FIG. 2A. As shown in FIG. 2A, larger orifices 215 are positioned proximate to fluid intake vent 155, smaller orifices 220 are positioned a selected distance from fluid intake vent 155, and slits 225 are positioned between the larger orifices 215 and the smaller orifices 220. In this embodiment of the present invention, orifices 215 and 220 provide greater mass flow rates of the secondary flows to first ends and second ends of light sources 110A, 110B, 110C and 110D (FIG. 1B) compared to the mass flow rates directed toward the lamp center regions of the light sources. This distribution of secondary flows provides preferential cooling to the external light source surfaces proximate to the cathodes. As a result of changes in momentum of the primary flow as it flows through the plenum chamber, an orifice having a large area positioned proximate to the flow actuator provides substantially the same mass flow rate as an orifice having a smaller area positioned a distance from the flow actuator. Therefore, the configuration illustrated in FIG. 2A provides substantially equivalent mass flow rates of the secondary flows to first ends and second ends of light sources 110A, 110B, 110C and 110D. In an embodiment of the present invention, the ratio of the area of larger orifices 215 and the area of smaller orifices 220 is selected over the range of about 1.1 to about 3.0. In one embodiment, the ratio of the area of larger orifices 215 and the area of smaller orifices 220 is equal to about 1.4. In an embodiment of the present invention, the ratio of the area of larger orifices 215 and the area of slits 225 is selected over the range of about 0.20 to about 0.99. In one embodiment, the ratio of the area of larger orifices 215 and the area of slits 225 is equal to about 0.66. As will be clear to one of skill in the art, the orifice dimensions provided here are meant to illustrate but one exemplary embodiment of the devices and methods of the present invention. Other orifice dimensions, particularly ratios of the areas of larger orifices to small orifices, are employable in the present invention, particular for embodiments having, other light source geometries, patterns of orifices, fan positions, and any combination of these variables.

FIG. 2B shows a top plan view of an alternative exemplary fluid distribution means comprising a fluid distribution plate 300, particularly useful for illuminators having light sources comprising mercury vapor fluorescent lamps having high energy producing cathodes. The fluid distribution plate 300 shown is characterized by a pattern of orifices which are symmetrical with respect to fluid distribution plate axis 205. The symmetrical pattern of orifices is especially useful for establishing a substantially uniform temperature profile along the external surfaces of light sources 110A, 110B, 110C and 110D (FIG. 1B) for an illuminator configuration having a flow actuator 170 and fluid intake vent 155 capable of generating a primary flow having a component parallel to fluid distribution plate axis 205. In one embodiment, light sources (not shown in FIG. 2B) are positioned along central axes 130A, 130B, 130C and 130D indicated in FIG. 2B. In the exemplary embodiment shown in FIG. 2B, the pattern of orifices includes orifices 315 and slits 325 positioned along central lamp axes 130A, 130B, 130C and 130D. In this embodiment of the present invention, orifices 315 provide substantially equivalent mass flow rates of the secondary flows to first ends and second ends of light sources (See FIG. 1A). Further, this pattern of orifices provides greater mass flow rates of the secondary flows to first ends and second ends of light sources 110A, 110B, 110C and 110D (FIG. 1A) proximate to cathodes compare to the mass flow rates directed at the center region.

The illuminators shown in FIGS. 1A and 1B can optionally be configured in a manner providing closed loop feedback temperature control for establishing and maintaining substantially constant radiant intensities and/or powers during treatment of a sample. In this aspect of the present invention, illuminator 100 further comprises a temperature sensor 420 and a temperature controller 425, as shown in FIG. 1A. Temperature sensor 420 is in operably coupled to one or more of light sources 110A, 110B, 110C and 110D and is capable of periodically generating output signals corresponding to the temperature of a detection area(s) located on one or more the external surfaces of the light sources 110A, 110B, 110C and 110D. In the exemplary embodiment shown in FIG. 1A, temperature sensor 420 is positioned on the external surface of fluid distribution means 160. In one embodiment, the temperature of detection area(s) of external surface of one or more of the light sources 110A, 110B, 110C and 110D is representative of the temperature profile of one or more of the light sources 110A, 110B, 110C and 110D along their central axis 130A, 130B, 130C and 130D. Temperature controller 425 is in operably coupled to temperature sensor 420 and receives the output signals corresponding to the temperature of the detection area(s) of external surface of one or more of the light sources 110A, 110B, 110C and 110D. Temperature controller 425 is operably coupled to the selectively adjustable flow actuator 170 and can selectively adjust the flow actuator 170 to provide a mass flow rate of the primary flow 157 which establishes secondary flows 165 necessary to maintain a substantially constant temperature of the detection area(s) of external surface of one or more of the light sources 110A, 110B, 110C and 110D equal to a pre-selected set point temperature. Secondary flows exit illuminator 100 through exhaust vents 185 resulting in a net transfer of thermal energy from the illuminator to the ambient surroundings.

In this aspect of the present invention, the temperature of the illuminator 100 is controlled by closed loop feedback temperature control. The rate of forced convection cooling in this embodiment of the present invention is principally determined by the mass flow rate of the primary flow in plenum chamber 150, which establishes the mass flow rates of secondary flows directed at the light source external surfaces. Temperature control is provided utilizing a flow actuator capable of generating a mass flow rate of the primary flow in the plenum chamber which is selectively adjustable. Closed loop feedback temperature control is provided by adjusting the mass flow rate of the primary flow on the basis of periodic in situ temperature measurements.

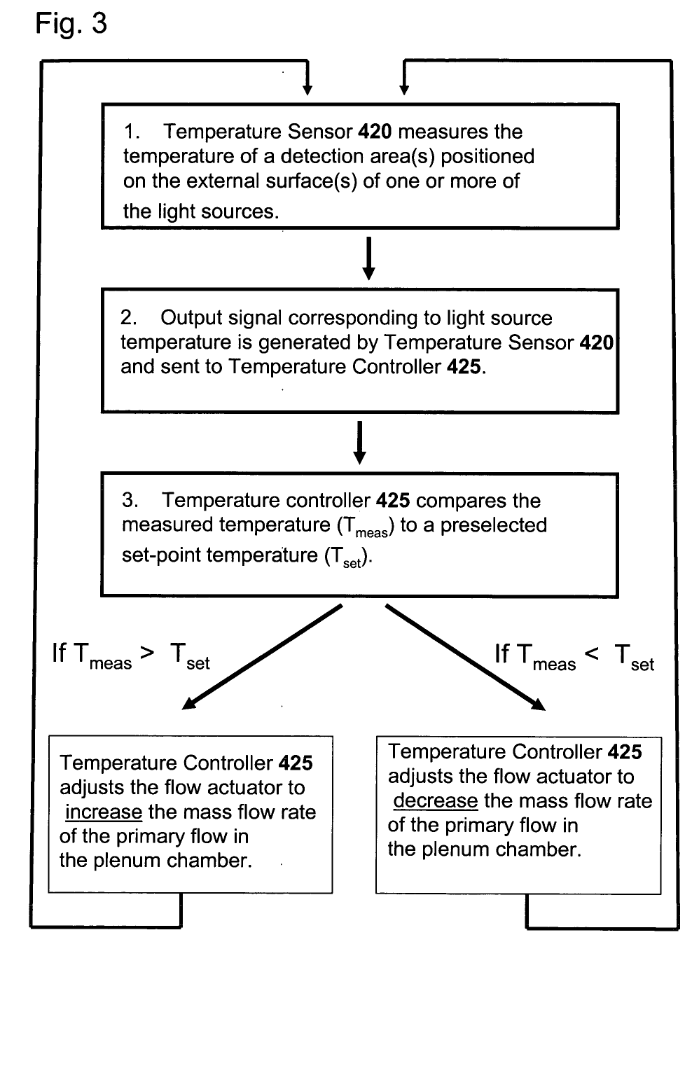

FIG. 3 provides a flow chart illustrating a closed loop feedback temperature control method useful for maintaining substantially constant radiant intensities and radiant powers of an illuminator of the present invention. The temperature of a detection area representative of the temperature of one or more light sources is periodically measured by temperature sensor 420. The temperature sensor generates output signals corresponding to the measured temperature and sends the output signal to temperature controller 425. The temperature controller compares the measured temperature to a preselected set-point temperature. In the event that the measured temperature and the set point temperature differ by a pre-selected percentage deviation, the temperature controller adjusts the flow actuator to provide a mass flow rate of the primary flow necessary to establish and maintain the set point temperature. For example, if the measured temperature is greater than the set point temperature, the temperature controller adjusts the flow actuator to increase the mass flow rate of the primary flow in the plenum chamber. On the other hand, if the measured temperature is less than the set point temperature, the temperature controller adjusts the flow actuator to decrease the mass flow rate of the primary flow in the plenum chamber. As shown by the arrows in FIG. 3, this aspect of the invention provides an iterative method wherein the temperature of the light sources is monitored over time and adjustments are periodically made to the mass flow rate of the primary flow in the plenum chamber based on these in situ temperature measurements to establish and maintain substantially constant radiant intensities and radiant powers.

As will be understood by one of skill in the art of feedback control theory, any proportional control algorithm can be used in the methods and devices of the present invention and the description provided here is intended to be merely illustrative of but one exemplary embodiment and not intended to be limiting in any way. As the radiant output of many light sources, such as mercury vapor fluorescent lamps, is dependent on lamp surface temperature, the closed loop feedback temperature control methods of this embodiment of the present invention provide a means of controlling the radiant output of an illuminator having one or more mercury vapor fluorescent lamp. In an exemplary embodiment, the closed loop feedback temperature control methods of the present invention provide a means of maintaining substantially constant radiant intensities.

Optionally, illuminator 400 further comprises irradiance detector 475 for providing a complementary means of closed loop feedback temperature feedback control. Irradiance detector is optically coupled to one or more of light sources 110A, 110B, 110C and 110D. In the exemplary embodiment shown in FIG. 3, irradiance detector 475 is positioned on the external surface of fluid distribution means 160. Irradiance sensor 475 detects the radiant power of one or more of light sources 110A, 110B, 110C and 110D and periodically generates output signals corresponding to the measured radiant power and/or radiant intensity. Temperature controller 425 is operably connected to irradiance detector 475 such that it is capable of controlling lamp temperature in a manner which establishes a pre-selected set point radiant power and/or radiant intensity. For example, temperature controller 425 is configured to compare the measured radiant power to a pre-selected, set point radiant power and/or radiant intensity and adjust flow controller 445 to establish a mass flow rate in plenum chamber 150, which establishes a lamp temperature corresponding to the set point radiant power and/or radiant intensity. In this aspect of the invention, closed loop feedback temperature control is achieved in a manner providing optimal irradiance conditions for sample treatment.

In one embodiment, light sources useable in this aspect of the present invention comprise a plurality of light sources, such as a plurality of tubular mercury vapor fluorescent lamps. This embodiment of the present invention employs a single temperature sensor capable of monitoring the temperature of a detection area on a single light source, such as a detection area which is representative of the temperatures of the additional light sources of the multiple light source illuminator. Alternatively, an embodiment of the present invention having a plurality of light sources has a plurality of temperature sensors. In this embodiment, the temperature controller is capable of averaging the output signals from each of the temperature sensors, comparing the average temperature to the set point temperature and adjusting the flow actuator to establish and maintain an average temperature equal to the set point temperature.

Temperature sensors useable in the present invention include any device or device component capable of accurately monitoring the external surface temperature of a light source. A non-contact temperature sensor, such as an infrared temperature sensor, is useful in some applications because it does not significantly affect the external surface temperature and, hence, does not influence the radiant intensities generated by the light source. An exemplary temperature sensor useable in the present invention is a thermopile infrared sensor. Alternatively, temperature sensors of the present invention are in physical contact and/or thermal contact with one or more external surfaces of light sources of the present invention. Exemplary temperature sensors for use in the present invention are capable of generating output signals corresponding to the temperature of the detection area having an area of about 20 mm² and at a rate of about 1 s⁻¹.

Selectively adjustable flow actuators of the present invention are capable of generating a primary flow in the plenum chamber having a selectively adjustable mass flow rate. Exemplary flow actuators include, but are not limited to, variable speed fans and variable pumping rate fluid pumps. In an exemplary embodiment, the mass flow rate established by the selectively adjustable flow actuator is established by the input voltage applied to the actuator.

Temperature controllers of the present invention include but are not limited to microprocessors and microcomputers. For example, a microprocessor or microcomputer uses a proportional control algorithm capable of calculating the magnitude of the increase or decrease in primary fluid flow needed to establish and maintain a selected set point temperature. In one embodiment, the set point temperatures used in the present invention are constant, pre-selected set point temperatures. Alternatively, the set point temperatures are variable and, thus, be selectively adjustable for a given application or sample undergoing treatment. Further, the pre-selected set point temperature of this aspect of the present invention can be selected to provide the optimal temperature for light source operation. For example, a set point temperature can be selected which provides the lowest current through a mercury vapor fluorescent lamp necessary to provide a desired radiant power, the optimal radiant output stability, the optimal radiant output power, the optimal distribution of emission wavelengths for a given photosensitive compound or combination of photosensitive compounds or any combination of these parameters which is best suited for a selected application or biological sample undergoing treatment.

Illuminators of the present invention comprise a single light source or a plurality of light sources. In an exemplary embodiment, two light sources are optically coupled to a sample chamber to provide illumination from planes above and below the sample. An advantage of using a plurality of light sources for sample treatment is that these optical configurations are capable of delivering very large intensities to the sample undergoing treatment.

The present invention provides methods, devices and device compounds for treating samples with electromagnetic radiation, especially useful for reducing pathogens in biological samples. As will be recognizable to those having skill in the art, all devices, device elements and device equivalents are within the scope of the present invention. The present invention provides exemplary illuminators having substantially constant radiant intensities and emission wavelengths, which provide for the reproducible exposure of biological samples to substantially constant radiant powers. In addition, the present invention provides illuminators having reduced temperature gradients, which provides enhanced lamp longevity, particularly for fluorescent lamp light sources. These and other variations of the present illuminators and methods of sample illumination are within the spirit and scope of the claimed invention. Accordingly, it must be understood that the detailed description, exemplary embodiments, drawings and examples set forth here are intended as illustrative only and in no way represent a limitation on the scope of the invention.

### EXAMPLE 1

### Illuminator for Treatment of Blood Component Samples with Light Having Wavelengths in the Ultraviolet Region

It is a goal of the present invention to provide ultraviolet light illuminators capable of delivering substantially constant ultraviolet light radiant intensities and powers to blood component samples. Further, it is a goal of the present invention to provide illuminators exhibiting reduced fluorescent lamp temperature gradients and having improved lamp longevities.

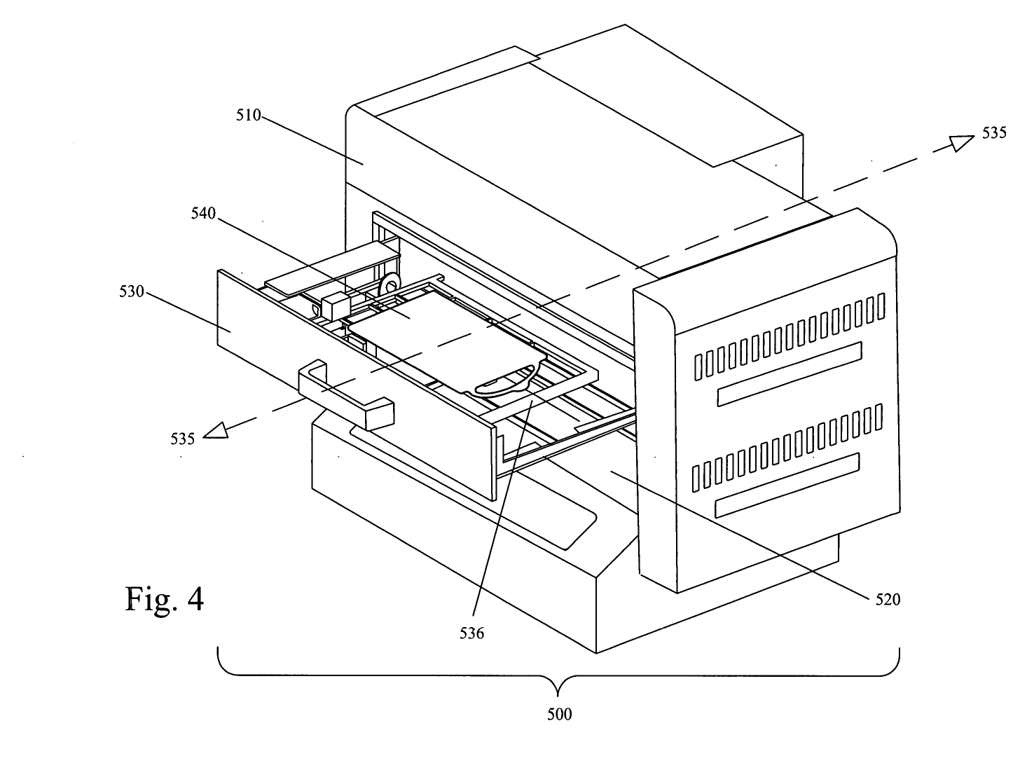

FIG. 4 is a schematic drawing showing an exemplary ultraviolet light illuminator having differential cooling and closed loop feedback temperature control. The ultraviolet light illuminator 500 comprises a first differentially cooled light source 510 and a second differentially cooled light source 520. First and second differentially cooled light sources are substantially identical and are positioned in optical communication with a sample compartment 530 such that a sample undergoing treatment is illuminated from planes both above and below the sample compartment 530. In the exemplary embodiment shown in FIG. 4, sample compartment 530 is selectively positionable along sample alignment axis 535 and is equipped with a sample agitator 536. Fluid container 540 is also shown in FIG. 4, and can be operably connected to sample compartment 530 by any means known in the art. Fluid container 540 contains the sample undergoing treatment. In some embodiments, sample agitation in treatment of liquid and colloidal samples ensures all portions of a sample are uniformly exposed to the same radiant powers.

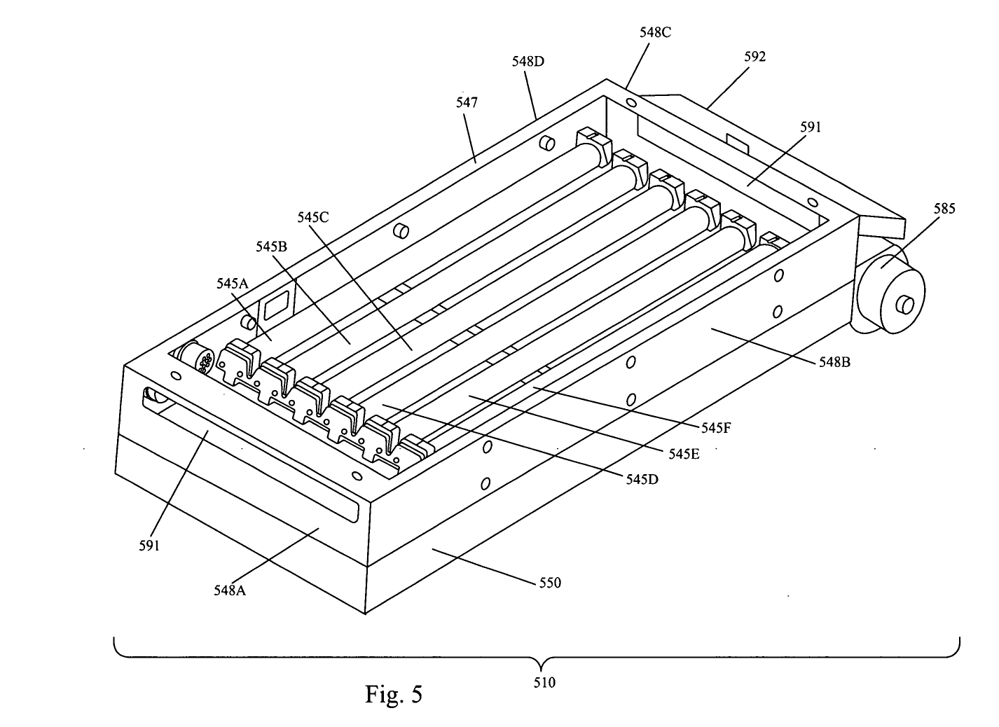

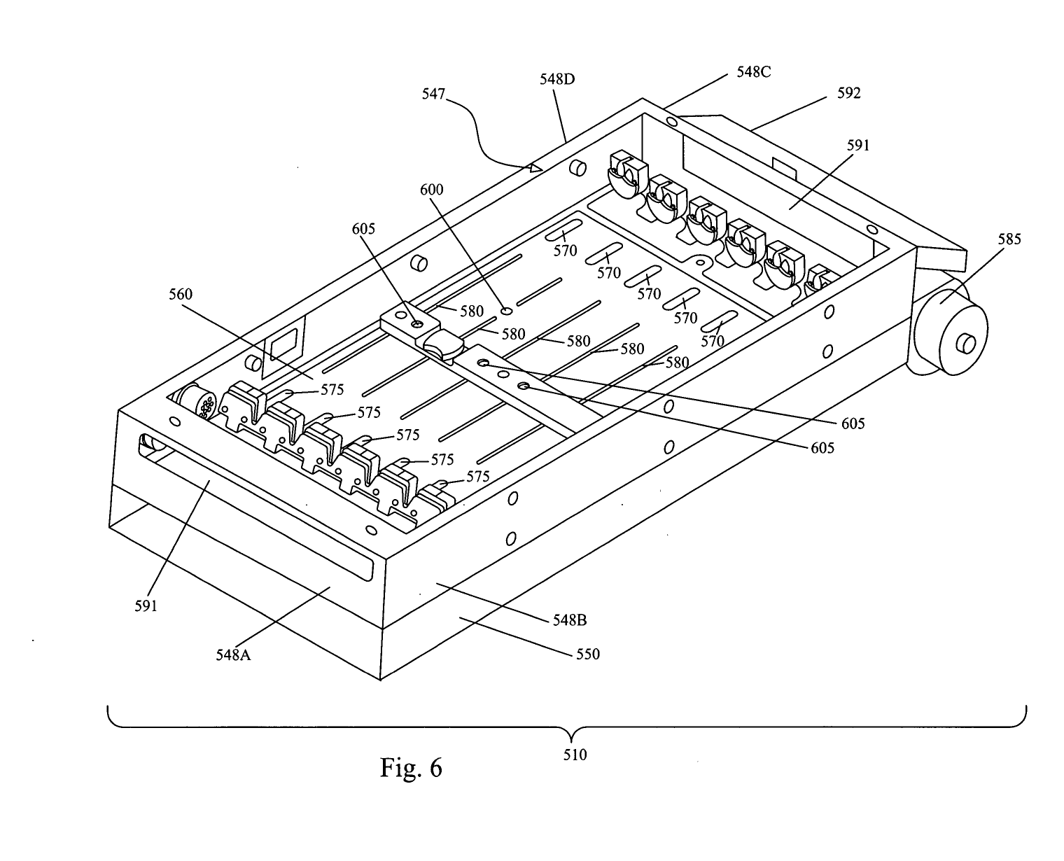

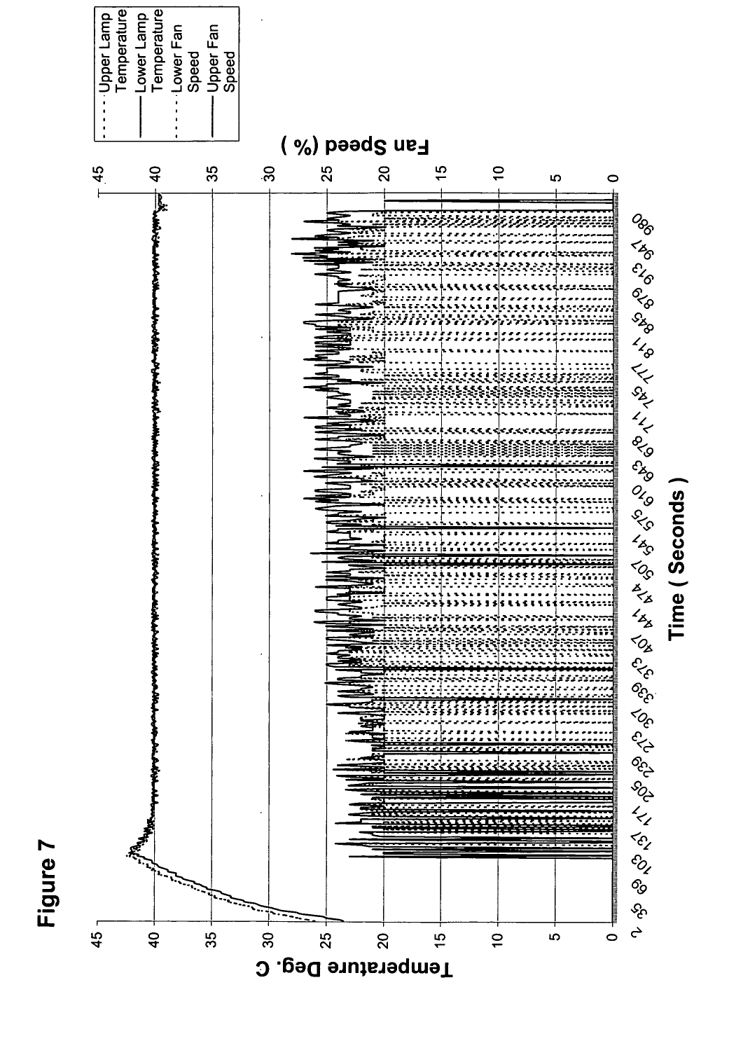

FIG. 5 is a schematic drawing of first differentially cooled light source 510 comprising 6 tubular fluorescent lamps 545A, 545B, 545C, 545D, 545E and 545F positioned in lamp housing 547 having sides 548A, 548B, 548C and 548D. Fluorescent lamps 545A, 545B, 545C, 545D, 545E and 545F are arranged in a parallel geometry wherein adjacent lamps are positioned about 3 cm from each other's central longitudinal axes. In the embodiment illustrated in FIG. 5, fluorescent lamps have diameters equal to about 25 mm and extend about 44 cm along there respective central longitudinal axes. In an exemplary embodiment, fluorescent lamps 545A, 545B, 545C, 545D, 545E and 545F each have current draws greater than about 450 mA. Also shown in FIG. 5 are flow actuator 585 operably connected to intake vent (not shown in the perspective view illustrated in FIGS. 6 and 7), exhaust vents 591 and flow partition 592. Flow actuator 585 shown in FIG. 5 is a variable speed fan. Referring again to FIG. 5, optionally a flow diffuser (not shown in the perspective view illustrated in FIGS. 6 and 7) is also provided in fluid communication with flow actuator 585 FIG. 6 is a schematic of first differentially cooled light source 510 with the fluorescent lamps removed from view. As shown in FIG. 6, first differentially cooled light source 510 also comprises plenum chamber 550 having an intake vent (not shown in FIGS. 4-6) and fluid distribution plate 560. Fluid distribution plate 560 is positioned proximate to the external surfaces of lamps 545A, 545B, 545C, 545D, 545E and 545F, for example positioned at a distance from the external surfaces selected from the range of about 0.5 cm to about 3 cm. In one embodiment, fluid distribution plate 560 is positioned about 2.5 centimeters from the external surfaces of lamps 545A, 545B, 545C, 545D, 545E and 545F. Fluid distribution plate 560 has a plurality of orifices including a first set of 4.0 cm² obrotund orifices 570 positioned about 10 cm from the fluid intake vent, a second set of 2.9 cm² obrotund orifices 575 positioned about 43 cm from fluid intake vent and 6.1 cm² slots 580 disposed between said first and second sets of obrotund orifices. In an exemplary embodiment, the area of the intake vent is approximately equal to the cumulative areas of first obrotund orifices 570, second obrotund orifices 575 and slots 580.

First differentially cooled light source 510 also includes flow actuator 585 and a flow diffuser. In one embodiment, flow diffuser is operably coupled to the intake vent and comprises a 0.15 cm thick plate with a plurality of 0.4 cm diameter holes with a 0.5 cm staggered spacing resulting in a porosity of approximately 63%. In the embodiment illustrated in FIGS. 4-6, the flow diffuser and flow actuator 585 are coupled to the intake vent in a manner providing a primary flow into plenum chamber 550. Lamp housing 547 is also equipped with exhaust vent 591 positioned on a side opposing the flow actuator 585, which in one embodiment is a variable speed fan, and a flow partition 592 to prevent exhaust fluid from being re-circulated into the differential cooling system.

First and second differentially cooled light sources are also equipped with closed loop feedback temperature control systems to ensure that radiant intensities and powers are substantially constant during sample illumination. In an exemplary embodiment, non-contact, temperature sensor 600 is positioned in lamp housing 547 and provides measurements of the temperature of the external surface of a selected fluorescent lamp. In the exemplary embodiment shown in FIG. 6, temperature sensor 600 is positioned on the external surface of fluid distribution plate 560. In alternative embodiments, one or more temperature sensors are located on any of sides 548A, 548B, 548C and 548D of lamp housing 547 or on the external surfaces of lamps 545A, 545B, 545C, 545D, 545E and 545F (see FIG. 5). In an exemplary embodiment, temperature sensor 600 is a MLX90601EZA model number thermopile infrared temperature sensor manufactured by Melexis Microelectronic Integrated Systems and provides temperature measurements at a rate of 1 s⁻¹. Use of an infrared temperature detector is useful for some applications because it is not in physical contact with the lamp surface, and, therefore, does not affect the temperature or temperature profile of the lamp during temperature monitoring and control. To provide closed loop feedback temperature control, temperature sensor 600 are operably connected to a temperature controller (not shown in FIGS. 4-6), such as a microprocessor or computer, capable of controlling the primary mass flow rate generated by the flow actuator 585. The exemplary UV illuminator 510, also comprises irradiance detectors 605 positioned in lamp housing 547, which provide redundant monitoring of the radiant intensities and powers generated by the fluorescent lamps. As shown in FIG. 6, irradiance detectors 605 are positioned on a support operably connected to fluid distribution plate 560 and monitor the intensity of light originating from sides of fluorescent lamps 545A, 545B, 545C, 545D, 545E and 545F which are opposite the sample undergoing treatment. In one embodiment, irradiance detectors 605 are operably connected to a temperature controller (not shown in FIGS. 4-6), such as a computer or microprocessor, to provide closed loop temperature control of the ultraviolet light illuminator 500 (See FIG. 4) using control signals derived from measurements of radiant power.

FIG. 7 shows a plot of lamp temperatures and fan speeds versus time observed for the exemplary ultraviolet light illuminator illustrated in FIGS. 4-6. As shown in FIG. 7, a steady state temperature equal to about 40° C. is established within approximately 137 seconds for both first and second differentially cooled light sources 510 and 520. Upon reaching steady state, the temperature is observed to be constant to within about 0.1° C. through the entire 9-minute sample exposure time. The temperature profile shown in FIG. 7 shows that ultraviolet illuminators of the present invention rapidly achieve steady state lamp temperatures useful for blood processing applications. In addition, the degree of temperature stability shown in FIG. 7 illustrates the effectiveness of the closed loop feedback temperature control methods of the present invention.

### 2. Illuminator for Treatment of Blood Component Samples with Light Having Wavelengths in the Visible region

It is a goal of the present invention to provide visible light illuminators capable of delivering substantially constant visible light radiant intensities and powers to blood component samples. Further, it is a goal of the present invention to provide illuminators exhibiting reduced fluorescent lamp temperature gradients and having improved lamp longevities.

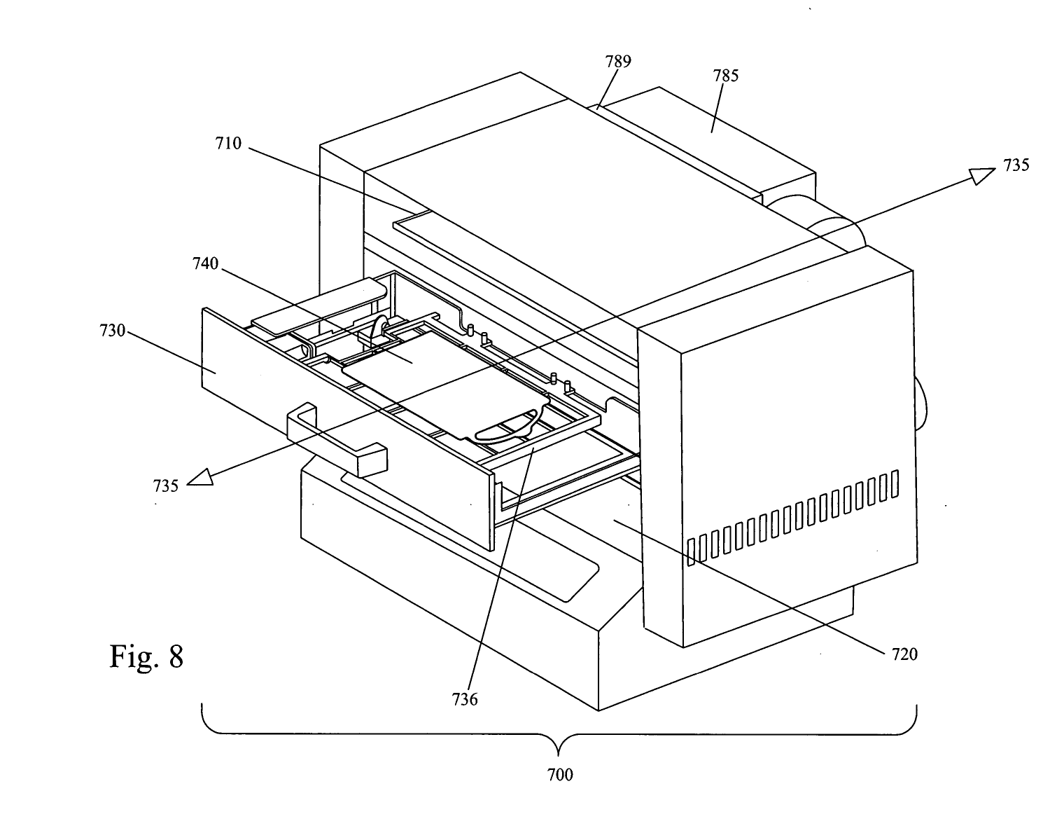

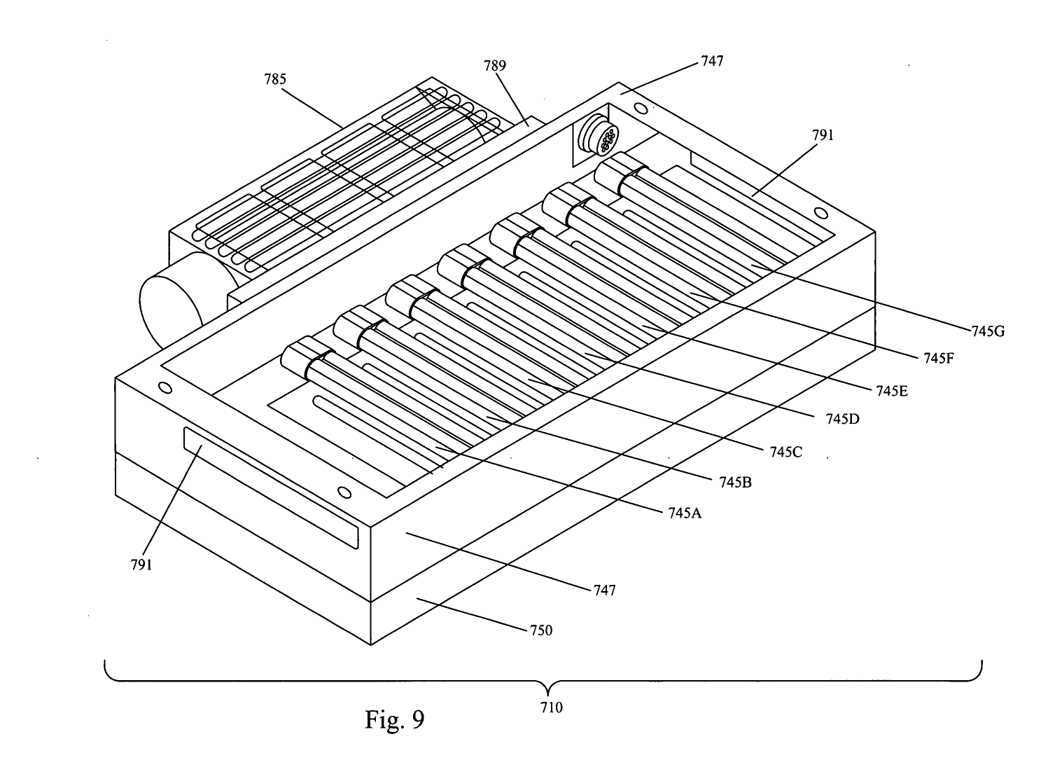

FIG. 8 is a schematic drawing showing an exemplary visible light illuminator having differential cooling. The visible light illuminator 700 comprises a first differentially cooled light source 710 and a second differentially cooled light source 720. First and second differentially cooled light sources 710 and 720 are substantially identical and are positioned in optical communication with a sample compartment 730 such that a sample undergoing treatment is illuminated from planes both above and below the sample compartment. In the exemplary embodiment shown in FIG. 8, sample compartment 730 is selectively positionable along sample alignment axis 735 and is equipped with a sample agitator 736. Fluid container 740 is also shown in FIG. 4, and can be operably connected to sample compartment 730 by any means known in the art. Fluid container 740 contains the sample undergoing treatment.

FIG. 9 is a schematic drawing of first differentially cooled light source 710 comprising 7 U-shaped tubular fluorescent lamps 745A, 745B, 745C, 745D, 745E, 745F and 745G positioned in lamp housing 747. As shown in FIG. 9, fluorescent lamps 745A, 745B, 745C, 745D, 745E, 745F and 745G are arranged in a parallel geometry wherein adjacent lamps are positioned about 6 cm from each other's center line. Fluorescent lamps extend about 30 cm along their respective central lamp axes. In an exemplary embodiment, the fluorescent lamps 745A, 745B, 745C, 745D, 745E, 745F and 745G are mercury vapor fluorescent lamps and have a current draw greater than about 450 mA.

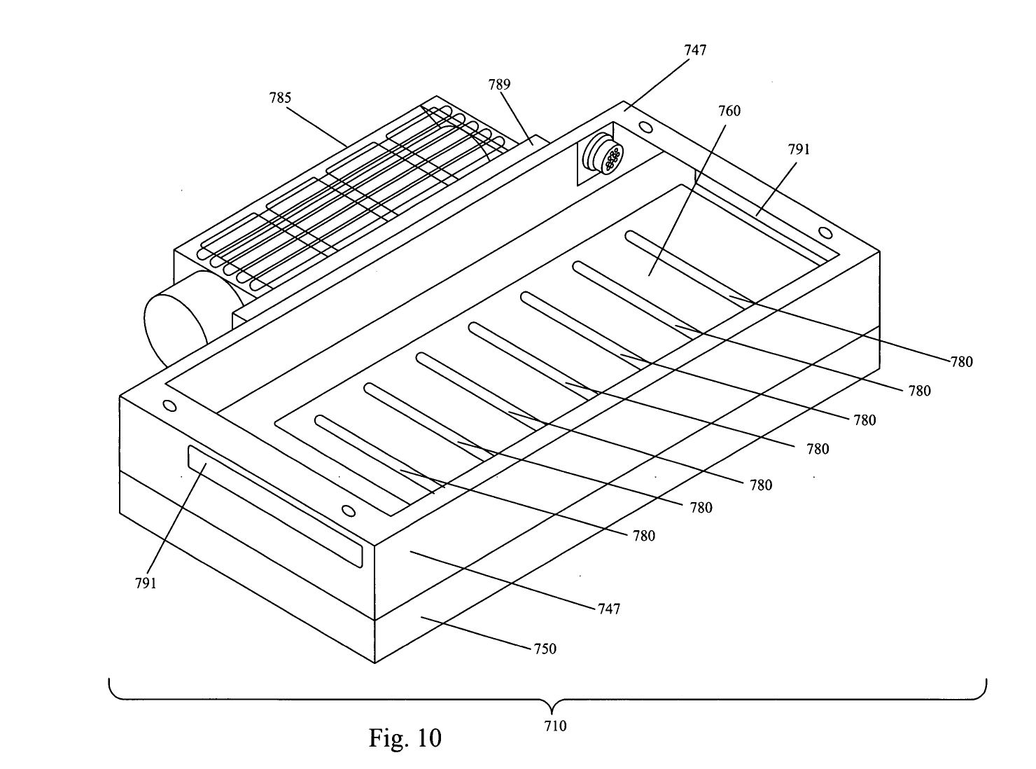

FIG. 10 is a schematic of first differentially cooled light source 710 with the fluorescent lamps removed. As shown in FIG. 10, first differentially cooled light source 710 comprises plenum chamber 750 having a fluid intake vent (not shown in perspective view of FIG. 10) and fluid distribution plate 760. Fluid distribution plate 760 is positioned proximate to the external surfaces of the lamps and has a plurality of orifices, for example positioned a distance from the external surfaces selected from the range of about 0.5 to about 3 cm. In one embodiment, fluid distribution plate 760 is positioned 2.5 cm from the external surfaces. In the exemplary embodiment shown, 16 cm² slots 780 extend along axes corresponding to the central longitudinal axis of each lamp. First differentially cooled light source 710 also includes flow actuator 785. In the embodiment illustrated in FIGS. 8-10, flow actuator 785 is coupled to the intake vent in a manner providing a primary flow into plenum chamber 750. Referring again to FIGS. 9 and 10, optionally flow diffuser 789 comprising a plate having a plurality of orifices is also provided in fluid communication with flow actuator 585 and the intake vent in a manner providing a primary flow into plenum chamber 550. Lamp housing 747 is also equipped with two exhaust vents 791 positioned on two opposing sides of the housing to ensure that air exiting the lamp housing is not drawn back into the fluid intake vent.

All references cited in this application and all references cited in these references are hereby incorporated in their entireties by reference herein to the extent that they are not inconsistent with the disclosure in this application. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques specifically described herein are intended to be encompassed by this invention.

1. An illuminator for treating a biological sample, said illuminator comprising:
a light source extending along a central lamp axis; and
a differential cooling system comprising a plenum chamber in fluid communication with said light source having a fluid intake vent for generating a primary flow into said plenum chamber and a fluid distribution means proximate to said light source having a plurality of orifices, wherein said fluid distribution means generates a distribution of secondary flows directed toward an external surface of said light source,
whereby the area, position, and shape of said orifices or any combination of these is selected such that the external surface of said light source has a desired temperature profile along said central lamp axis.

2. The illuminator of claim 1, wherein said light source has a first end, a second end, and a central region positioned between said first end and said second end, wherein said first end is proximate to a first cathode of said light source and said second end is proximate to a second cathode of said light source.

3. The illuminator of claim 1, wherein said light source has a substantially uniform temperature profile along said central lamp axis.

4. The illuminator of claim 1, wherein said primary flow has a component which flows in a direction substantially parallel to said central lamp axis.

5. The illuminator of claim 1 wherein said primary flow has a component which flows in a direction substantially orthogonal to said central lamp axis.

6. The illuminator of claim 2, wherein said distribution of secondary flows comprises a plurality of secondary flows directed at said first end, said second end and said central region.

7. The illuminator of claim 6, wherein said distribution of secondary flows comprises a first secondary flow directed at said first end, a second secondary flow directed at said second end and a third secondary flow directed at said central region.

8. The illuminator of claim 7, wherein said first and second secondary flows are each greater than said third secondary flow.

9. The illuminator of claim 1, wherein said orifices are positioned at selected points along the central lamp axis of said light source.

10. The illuminator of claim 1, wherein said fluid intake vent has an area substantially equal to the cumulative area of said orifices.

11. The illuminator of claim 1, wherein said fluid distribution means comprises a fluid distribution plate, wherein said fluid distribution plate has an internal side and an external side, wherein said orifices extend through said internal and external sides of said fluid distribution plate and wherein said external side is positioned proximate to said light source.

12. The illuminator of claim 11, wherein said plurality of orifices comprises at least one first end orifice, at least one central region orifice and at least one second end orifice.

13. The illuminator of claim 12, wherein said first end orifice is positioned closer to said intake vent than said second end orifice along said central lamp axis.

14. The illuminator of claim 13, wherein the area of said first end orifice is greater than the area of said second end orifice.

15. The illuminator of claim 14, wherein the ratio of the area of said first end orifice and the area of the said second end orifice is selected from the range of about 1.1 to about 3.0.

16. The illuminator of claim 12, wherein said first end orifice and said second end orifice are substantially obrotund shaped.

17. The illuminator of claim 12, wherein said first end orifice and said second end orifice are substantially circular.

18. The illuminator of claim 12, wherein the area of the first end orifice is smaller than the area of the central region orifice.

19. The illuminator of claim 18, wherein the ratio of the area of the first end orifice and the area of the central region orifice is selected from the range of about 0.20 to about 0.99.

20. The illuminator of claim 12, wherein said central region orifice is a slit extending along said central lamp axis.

21. The illuminator of claim 11, wherein said external side of said fluid distribution plate is highly reflective.

22. The illuminator of claim 11, wherein said external side of said fluid distribution plate is substantially flat.

23. The illuminator of claim 11, wherein said external side of said fluid distribution plate is substantially curved.

24. The illuminator of claim 11, wherein said external side of said fluid distribution plate is positioned a distance from the external surface of said light source selected from the range of about 0.3 cm to about 3 cm.

25. The illuminator of claim 1, wherein said differential cooling system further comprises a flow actuator connected to said fluid intake vent for generating said primary flow.

26. The illuminator of claim 1 wherein said differential cooling system further comprises a flow diffuser in fluid communication with said intake vent.

27. The illuminator of claim 26, wherein said flow diffuser is a porous screen.

28. The illuminator of claim 25 wherein said flow actuator comprises a fan.

29. The illuminator of claim 25 wherein said flow actuator comprises a fluid pump.

30. The illuminator of claim 1, further comprising additional light sources each extending along additional central lamp axes oriented parallel to said central lamp axis, wherein each additional light source is in fluid communication with said plenum chamber and wherein said fluid distribution means generates a distribution of secondary flows directed toward the external surfaces of said additional light sources.

31. The illuminator of claim 30, wherein the area, position, and shape of said orifices or any combination of these is selected such that the surfaces of said additional light sources have desired temperature profiles along said additional lamp axes.

32. The illuminator of claim 31, wherein said additional light sources have substantially uniform temperature profiles along said additional lamp axes.

33. The illuminator of claim 1, wherein said light source is a mercury vapor fluorescent lamp.

34. The illuminator of claim 1, wherein said light source generates visible light.

35. The illuminator of claim 1 wherein said light source is an array of light emitting diodes (LEDs).

36. The illuminator of claim 1, wherein said light source generates ultraviolet light.

37. The illuminator of claim 33, wherein said light source has a power selected from the range of about 10 mW cm⁻² to about 120 mW cm⁻².

38. The illuminator of claim 2, wherein said light source is a U-shaped fluorescent lamp having a bent region and wherein said central region is said bent region.

39. The illuminator of claim 1, wherein said fluid distribution means comprises a plurality of flow guides for directing said secondary flows toward the external surface of said light source.

40. A method of differentially cooling the surface of a light source for treating a biological sample, comprising the steps of:
generating a primary flow through a plenum chamber in fluid communication with the light source extending along a central lamp axis; and
passing at least a portion of said primary flow through a fluid distribution means having a plurality of orifices positioned proximate to said light source, thereby generating a distribution of secondary flows directed toward the surface of said light source, wherein the area, position and shape of said orifices or any combination of these is selected such that the surface of said light source has a desired temperature profile along said central lamp axis.

41. An illuminator for treating a biological sample, said illuminator comprising:
a light source;
a cooling system operably connected to said light source comprising:
a plenum chamber in fluid communication with said light source, said plenum chamber having a fluid intake vent and a fluid distribution means; and
a selectively adjustable flow actuator in fluid communication with said fluid intake vent for generating a primary flow into said plenum chamber,
wherein said fluid distribution means generates at least one secondary flow directed toward an external surface of said light source for convectively cooling said external surface;
a temperature sensor operably coupled to said light source for generating an output signal corresponding to the temperature of the external surface of said light source; and
a temperature controller operably connected to said temperature sensor and said selectively adjustable flow actuator, wherein said temperature controller receives said output signal from said temperature sensor, compares said output signal to a set point temperature and adjusts the flow actuator to provide a primary fluid mass flow rate which establishes and maintains a substantially constant temperature of the external surface of said light source equal to said set point temperature.

42. The illuminator of claim 41, wherein said temperature sensor is not in physical contact with the external surface of said surface of said light source.

43. The illuminator of claim 41, wherein said temperature sensor detects infrared light generated by said light source.

44. The illuminator of claim 43, wherein said temperature sensor is a thermopile infrared sensor.

45. The illuminator of claim 41 wherein said temperature sensor is positioned on said fluid distribution means.

46. The illuminator of claim 41 wherein said temperature sensor is positioned proximate to said biological sample.

47. The illuminator of claim 41, wherein said flow actuator is a fan having a variable fan speed.

48. The illuminator of claim 41, wherein said flow actuator is a fluid pump having a variable pumping rate.

49. The illuminator of claim 41, wherein said light source is selected from the group consisting of:
a mercury vapor fluorescent lamp;
a cold cathode fluorescent lamp;
an excimer lamp;
a LED array;
an arc discharge lamp; and
a tungsten filament lamp.

50. The illuminator of claim 41, wherein said temperature sensor detects the temperature of a detection area on said light source and the temperature of said detection area is representative of the average temperature of said light source.

51. The illuminator of claim 41 wherein said set point temperature is about 40° C.

52. The illuminator of claim 41, wherein said set point temperature is selected to provide a minimum current through said light source.

53. A method for establishing and maintaining a substantially constant external surface temperature profile of a light source for treating a biological sample, comprising the steps of:
measuring the temperature of a detection area located on the external surface of said light source, said light source being operably connected to a cooling system comprising:
a plenum chamber in fluid communication with said light source, said plenum chamber having a fluid intake vent and a fluid distribution means; and
a selectively adjustable flow actuator operably connected to said fluid intake vent for generating a primary flow into said plenum chamber,
wherein said fluid distribution means generates at least one secondary flow directed toward the surface of said light source for convectively cooling the external surface of said light source;
comparing said temperature of said detection area to a set point temperature; and
adjusting the flow actuator to provide a primary fluid mass flow rate which establishes and maintains a substantially constant temperature of the detection area equal to said set point temperature for the duration of the illumination of said sample.

54. An illuminator for treating a biological sample, said illuminator comprising:
a light source;
a cooling system operably connected to said light source comprising:
a plenum chamber in fluid communication with said light source, said plenum chamber having a fluid intake vent and a fluid distribution means; and
a selectively adjustable flow actuator operably connected to said fluid intake vent for generating a primary flow into said plenum chamber,
wherein said fluid distribution means generates at least one secondary flow directed toward an external surface of said light source for convectively cooling said external surface;
an irradiance detector in optical communication with said light source for measuring the radiant power of said light source and for generating an output signal corresponding to the radiant power of said light source; and
a temperature controller operably connected to said irradiance detector and said selectively adjustable flow actuator, wherein said temperature controller receives said output signal from said irradiance detector, compares said output signal to a set point radiant power and adjusts the flow actuator to provide a primary fluid mass flow rate which establishes and maintains a substantially constant, selected radiant power of said light source equal to said set point radiant power.

55. An illuminator for treating a biological sample, said illuminator comprising:
a light source in optical communication with said biological sample, said light source extending along a central lamp axis;
a differential cooling system comprising a plenum chamber in fluid communication with said light source and a selectively adjustable flow actuator for generating a primary flow into said plenum chamber, said plenum chamber having a fluid intake vent operably connected to said flow actuator and a fluid distribution means having a plurality of orifices proximate to said light source, wherein said fluid distribution means generates a distribution of secondary flows directed toward an external surface of said light source and wherein the area, position, and shape of said orifices or any combination of these is selected such that the surface of said light source has a substantially uniform temperature profile along said central lamp axis;
a temperature sensor in operably coupled to said light source which generates an output signal corresponding to a temperature of a detection area located on the surface of said tubular light source; and
a temperature controller operably connected to said temperature sensor and said selectively adjustable flow actuator, wherein said temperature controller receives said output signal from said temperature sensor, compares said output signal to a set point temperature and adjusts the flow actuator to maintain a substantially constant temperature of the detection area equal to said set point temperature.

56. An illuminator for treating a biological sample, said illuminator comprising:
a light source in optical communication with said biological sample, said light source extending along a central lamp axis;
a differential cooling system comprising a plenum chamber in fluid communication with said light source and a selectively adjustable flow actuator for generating a primary flow into said plenum chamber, said plenum chamber having a fluid intake vent operably connected to said flow actuator and a fluid distribution means having a plurality of orifices proximate to said light source, wherein said fluid distribution means generates a distribution of secondary flows directed toward an external surface of said light source and wherein the area, position, and shape of said orifices or any combination of these is selected such that the surface of said light source has substantially uniform temperature profile along said central lamp axis;
an irradiance detector in optical communication with said light source for measuring the radiant power of said light source and for generating an output signal corresponding to the radiant power of said light source; and
a temperature controller operably connected to said irradiance detector and said selectively adjustable flow actuator, wherein said temperature controller receives said output signal from said irradiance detector, compares said output signal to a set point radiant power and adjusts the flow actuator to maintain a substantially constant radiant power of the light source equal to said set point radiant power.
